(19) United States
(12) Patent Application Publication
(10) Pub. No.: US 2023/0130102 A1
(43) Pub. Date: Apr. 27, 2023

(54) Na/K-ATPase LIGANDS AND USE THEREOF FOR TREATMENT OF CANCER

(71) Applicant: Marshall University Research Corporation, Huntington, WV (US)

(72) Inventors: Zijian XIE, Huntington, WV (US); Moumita BANERJEE, Huntington, WV (US); Joseph SHAPIRO, Huntington, WV (US); Yingnyu GAO, Huntington, WV (US); Maosheng DUAN, Huntington, WV (US); Xuchai TAN, Huntington, WV (US)

(21) Appl. No.: 17/767,276

(22) PCT Filed: Oct. 8, 2020
(86) PCT No.: PCT/US2020/054795

§ 371 Date: Apr. 7, 2022

Related U.S. Application Data
(60) Provisional application No. 62/912,453, filed on Oct. 8, 2019.

(51) Int. Cl. C07D 311/86; C07D 498/04; C07F 9/655; C07D 327/08; C07D 491/147; C07C 49/84; C07C 49/83; A61P 35/00; A61P 13/08; A61P 35/04

(52) U.S. Cl. CPC C07D 311/86; C07D 498/04; C07F 9/65522; C07D 327/08; C07D 491/147; C07C 49/84; C07C 49/83; A61P 35/00; A61P 13/08; A61P 35/04

(57) ABSTRACT

Na/K-ATPase ligands are provided that comprise a compound of formula (I) and are capable of binding to the α1 Na/K-ATPase and decreasing the endocytosis of α1 Na/K-ATPase, such that expression of the α1 Na/K-ATPase is restored in the plasma membrane of cells and tumor growth and invasion is reduced. Pharmaceutical compositions are further provided that include a compound of formula (I) and a pharmaceutically-acceptable vehicle, carrier, or excipient. Methods of treating a cancer are further included and comprise administering to a subject an effective amount of a Na/K-ATPase ligand.

### RELATED APPLICATIONS

This application claims priority from U.S. Provisional Application Ser. No. 62/912,453, filed Oct. 8, 2019, the entire disclosure of which is incorporated herein by this reference.

### GOVERNMENT INTEREST

This invention was made with government support under grant number HTL109015 awarded by the National Institutes of Health. The government has certain rights in the invention.

### TECHNICAL FIELD

The presently-disclosed subject matter generally relates to Na/K-ATPase ligands and the use of those ligands for the treatment of cancer. In particular, certain embodiments of the presently-disclosed subject matter relate to Na/K-ATPase ligands capable of binding to the al Na/K-ATPase and decreasing the endocytosis of α1 Na/K-ATPase, such that expression of the al Na/K-ATPase is restored in the plasma membrane of cells and tumor growth and invasion is reduced.

### BACKGROUND

Prostate cancer (PCa) is the second most common type of cancer in males and is treatable if detected at early stages with a five-year survival rate of nearly 100%. However, at advanced stages when the cancer spreads to distant organs through metastasis, this survival rate drops to only about 27%. Androgen deprivation therapy (ADT) is the first line of therapy for advanced PCa, but a significant fraction of these tumors become resistant and progress to metastatic castration resistant state (CRPC) for which there is no effective cure. Because most carcinomas are of epithelial origin, tumor cells reactivate a developmental program called EMT (Epithelial-Mesenchymal Transition). Tumor cells undergoing EMT alter their apical-basal polarity and lose their adherens junctions by activating mesenchymal genes, which in turn transcriptionally repress cell adhesion molecules. This enables them to escape the stressful tumor microenvironment by assuming a motile fibroblast-like phenotype to invade the vasculature, and subsequently colonize at distant sites. The major hallmarks of EMT are a loss of adhesion junction and cell polarity proteins (E-cadherin, ZO-1/2, claudins and occludin) and gain of expression of mesenchymal genes (e.g. SNAIL, ZEB1/2, TWIST). Novel molecular targets and therapeutics focused on EMT are an attractive approach in the treatment of metastatic PCa, which accounts for nearly 90% of PCa patient deaths.

Na/K-ATPase α1 (NKA), a transmembrane ion pump and a fundamental signaling mechanism in cell proliferation and differentiation, may be one such target. Indeed, NKA expression at the plasma membrane is an important determinant of epithelial apical-basal polarity and maintenance of cell-cell adhesion junctions, a feature that is frequently lost in EMT. Consistently, reduced NKA subunit (a and 3) expression has been reported in association with EMT in both cell and animal models of fibrosis and carcinoma. It has been reported that NKA expression levels are inversely correlated with metastatic spread of prostate carcinomas. Genetically targeted loss of α1 NKA in PCa cells subsequently causes a metabolic switch from oxidative phosphorylation to aerobic glycolysis (Warburg effect) through Src kinase activation, and increased tumor volume in a mouse xenograft model. Clinically, NKA α1 expression is largely undetectable in bone metastatic lesions of PCa patients, indicative of translational potential of increasing NKA expression as a therapeutic approach. Collectively, these studies have therefore established a strong and clinically significant link between NKA expression and invasiveness/metastasis of prostate carcinoma. They also suggest that EMT secondary to decreased NKA expression could be targeted to decrease metastasis/invasiveness of prostate carcinoma.

Alteration of NKA cellular distribution in PCa cells is secondary to an increase of al NKA receptor endocytosis. This mechanism is NKA-specific and can be modified pharmacologically by modulating its receptor function. Cardiotonic steroids (CTS) are the archetypal and best-studied NKA ligands. They bind to and inhibit the enzymatic activity of NKA by stabilizing the protein in its E2P conformation. Because E2P represents an active conformation for Src and α1 NKA interaction, CTS such as ouabain are agonists of the receptor al NKA/Src complex. Accordingly, these compounds stimulate protein and lipid kinases, increase Reactive Oxygen Species (ROS) production and induce the endocytosis of α1 NKA.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

The presently-disclosed subject matter includes Na/K-ATPase ligands and the use of those ligands for the treatment of cancer. In particular, certain embodiments of the presently-disclosed subject matter include Na/K-ATPase ligands capable of binding to the α1 Na/K-ATPase and decreasing the endocytosis of α1 Na/K-ATPase, such that expression of the al Na/K-ATPase is restored in the plasma membrane of cells and tumor growth and invasion is reduced. In some embodiments, a Na/K-ATPase ligand comprises a compound having the following formula (I):

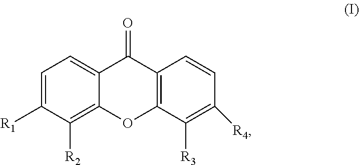

wherein R₁, R₂, R₃, and R₄ are independently selected from a hydrogen atom, a hydroxyl, an amine, a sulfonic acid, a thiol, a fluorine atom, or a phosphate group, or wherein R₁ and R₂ are a hydrogen and R₃ and R₄ are combined to produce a heterocyclic group including one or more nitrogen atoms.

In some embodiments, a compound of formula (I) comprises a compound having one of the following formulas (II)-(XIII):

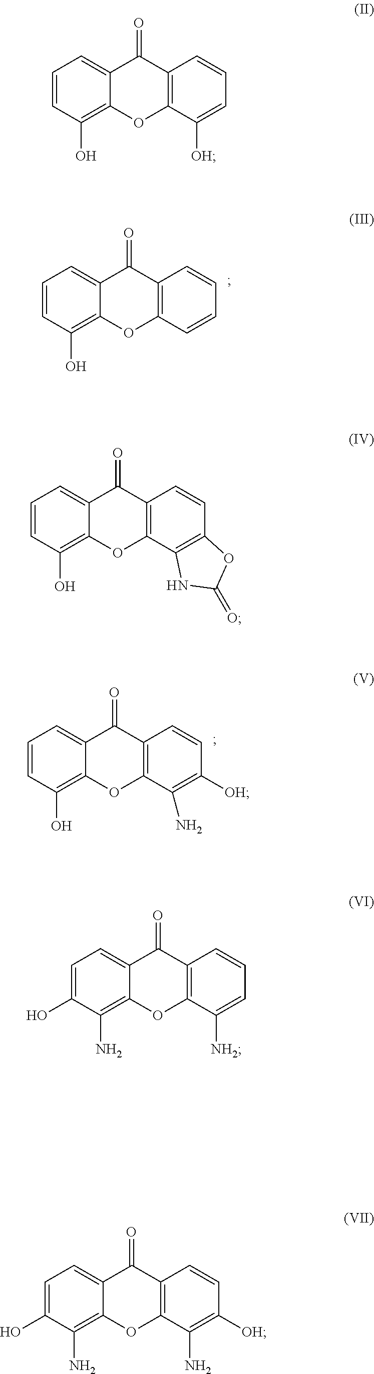 [SMILES: O=c1c2cccc(O)c2oc2c(O)cccc12 ; O=c1c2ccccc2oc2c(O)cccc12 ; O=c1[nH]c2c(ccc3c(=O)c4cccc(O)c4oc32)o1 ; Nc1c(O)ccc2c(=O)c3cccc(O)c3oc12 ; Nc1cccc2c(=O)c3ccc(O)c(N)c3oc12 ; Nc1c(O)ccc2c(=O)c3ccc(O)c(N)c3oc12]

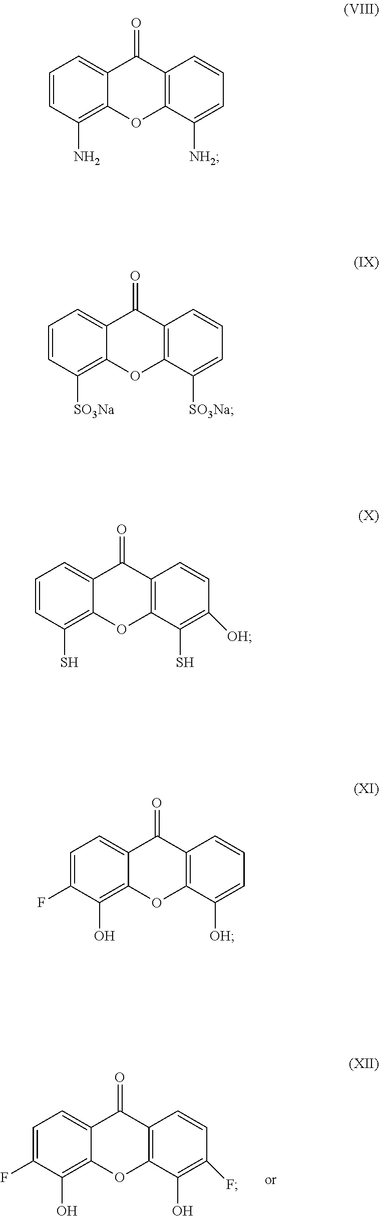

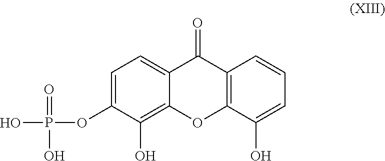 [SMILES: O=c1c2cccc(O)c2oc2c(O)c(OP(=O)(O)O)ccc12]

In some embodiments, a Na/K-ATPase ligand comprises a compound, having one of the following formulas:

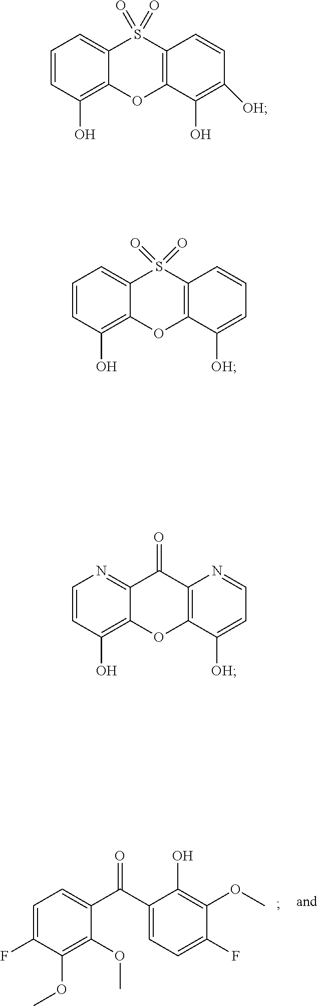 [SMILES: O=S1(=O)c2cccc(O)c2Oc2c1ccc(O)c2O ; O=S1(=O)c2cccc(O)c2Oc2c(O)cccc21 ; O=c1c2nccc(O)c2oc2c(O)ccnc12 ; COc1c(F)ccc(C(=O)c2ccc(F)c(OC)c2OC)c1O]

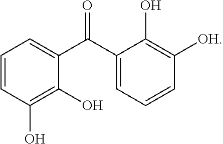 [SMILES: O=C(c1cccc(O)c1O)c1cccc(O)c1O]

Further provided, in some embodiments of the presently-disclosed subject matter are pharmaceutical compositions comprising a Na/K-ATPase ligand described herein and a pharmaceutically-acceptable vehicle, carrier, or excipient.

Still further provided, in some embodiments, are methods of treating a cancer that comprise administering to a subject an effective amount of a Na/K-ATPase ligand described herein. In some embodiments, the cancer is a primary cancer. In some embodiments, the cancer is a secondary cancer. In some embodiments, the subject has cancer and/or the Na/K-ATPase ligand (i.e., a compound of the presently-disclosed subject matter) is administered in an amount sufficient to reduce an endocytosis of an α1 Na/K-ATPase in a cancer cell. In some embodiments, such a Na/K-ATPase ligand or compound has the following formula (XIV) or the following formula (XV):

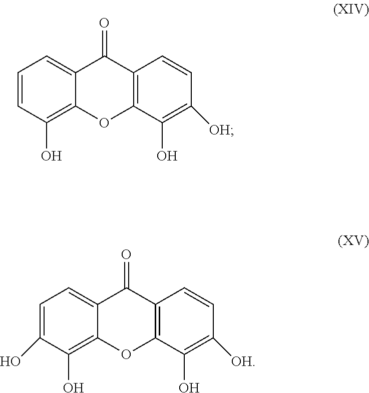 [SMILES: O=c1c2cccc(O)c2oc2c(O)c(O)ccc12 ; O=c1c2ccc(O)c(O)c2oc2c(O)c(O)ccc12]

Further features and advantages of the present invention will become evident to those of ordinary skill in the art after a study of the description, figures, and non-limiting examples in this document.

### BRIEF DESCRIPTION OF THE DRAWINGS

FIGS. 1A-1G include graphs and images showing the use of a Na/K-ATPase ligand, MB5, increases Na/K-ATPase α1 (NKA) expression in cells by preventing its endocytosis, including: (FIG. 1A) a diagram showing the chemical structure of MB5, the parent compound; (FIG. 1B-1C) graphs showing Na/K-ATPase activity assay of MB5 with purified pig kidney enzyme; (FIG. 1D) images showing immunostaining of phosphoERK, where pretreatment of LLC-PK1 cells with MB5 alone did not activate ERK (green fluorescence) significantly (upper panels), and where ouabain treatment activated ERK signal but pretreatment with MB5 significantly decreased ouabain-induced ERK signal (lower panels, quantitative data is shown, N=3-4, **p<0.01 and *p<0.05 compared with control (One way ANOVA)); (FIG. 1E) a graph showing MB5 did not affect EGF-induced ERK activation (N=3, **p<0.01 compared with Control, One way ANOVA); (FIG. 1F) a graph showing MB5 did not affect dopamine-induced activation of ERK (N=3, **p<0.01 compared with Control, One way ANOVA); and (FIG. 1G) a graph showing ouabain treatment induced α1 NKA internalization (endocytosis) in TCN-YFP al cells, where pretreatment with MB5 inhibited ouabain-induced α1 NKA endocytosis in a concentration dependent manner (quantitative data is shown, **p<0.01 compared with untreated, ##p<0.01 compared with only ouabain treated group. (One way ANOVA). n=3).

FIGS. 2A-2E include graphs and images showing MB5 prevents tumor invasion and growth of prostate cancer cells, including: (FIG. 2A) an image showing a representative Western blot showing α1 NKA and other epithelial marker expression in four prostate cancer cell lines (upper panels), where the middle panels show expression of mesenchymal markers in three of these cell lines, and where the lower panels show immunostaining images of cellular distribution of α1 NKA in these three cell lines; (FIG. 2B) graphs showing (left) immunohistochemistry analysis showing comparative expression of α1 NKA in normal prostate gland, primary tumor and bone metastatic lesions and (right) quantitative data showing paired tissue analysis of al NKA expression in adjacent normal tissue and primary tumor of prostate gland from the same patients, with a table showing numerical expression data of α1 NKA in normal tissue vs. primary tumor and metastatic lesions in three different types of cancer (prostate, breast and kidney) in patients as analyzed by immunohistochemistry; (FIG. 2C) a schematic diagram for generation of highly aggressive prostate cancer cell lines by xenografting DU145 or α1 NKA KD cells (˜50% knockdown) into NOD/SCID mice, with a Western blot showing relative α1 NKA expression in the generated clones; (FIG. 2D) graphs and phase contrast microscope images of xenograft derived cell lines (top panels), with the bottom left panels showing loss of epithelial markers (E-cadherin, ZO-1, ZO-2 and Occludin) but upregulation of mesenchymal markers (SNAIL and ZEB1) in low α1 NKA expressing clones 4 and 2 with respect to 5 (as detected by Western blot). n=3-4, and with the bottom right panel showing qPCR verification of EMT status of clones 4 and 2 as compared with clone 5 (*p<0.05 and **p<0.01 compared with 5 (same RNA) by One way ANOVA); and (FIG. 2E) images showing tumor spheroids from clones 5 and 2 were generated and then embedded into a 3D matrix made of Matrigel and Collagen, where spheroid invasion into 3D matrix was monitored through 12 hours to Day 3 by phase contrast microscopy, where clone 2 invaded into matrix by 12 hours whereas Clone 5 did not invade into matrix up to 3 days (N=6 spheroids per clone type).

FIGS. 3A-3F are graphs and images showing the ability of MB5 to reduce the invasive potential of prostate cancer cells by preventing α1 NKA endocytosis; including: (FIG. 3A) a graph showing MB5 treatment significantly reduced α1 NKA endocytosis of clone 4, where a representative Western blot is shown above and quantitative data is presented below (N=3. **p<0.01 compared with control (One way ANOVA)); (FIG. 3B) images and a graph showing MB5 treatment significantly reduced invasion of clone 2 spheroids in a dose-dependent manner, where images of spheroid invasion with MB5 treatment are shown above and quantitative analysis is shown below (N=6-8 spheroids per condition, **p<0.01 (One way ANOVA)); (FIG. 3C) a graph showing the results of a Boyden chamber assay showing that MB5 inhibits cell migration of clone 2 as effectively as other kinase inhibitors—PP2 (Src kinase inhibitor) and FAK inhibitor (Focal adhesion kinase inhibitor); (FIG. 3D) an image showing 100 nM MB5 treatment increased α1 NKA and E-cadherin expression but decreased expression of mesenchymal markers (SNAIL and ZEB1) along with myc (oncogene) and PCNA (cell proliferation marker); (FIG. 3E) a graph showing MB5 treatment (20 mg/kg/day) reduces tumor growth in nude mice xenografted with DU145 or KD cells, where quantitative comparison of tumor weight from DU145 and KD cells are shown in upper panel (*p<0.05 as indicated (Students' t test), N=6-10 each group), where MB5 treatment (10 mg/kg) significantly reduced xenografted tumor growth from highly aggressive clones 4 and 2, derived from DU145 (lower panel), where cells were injected into both flanks of nude mice and after 4 weeks, daily MB5 injection was administered peritoneally, where tumor growth was assessed by scalpel twice every week, and where quantitative analysis of growth in tumor volume in presence or absence of MB5 treatment is shown (n=10 mice for each group. **p<0.01 and *p<0.05 as indicated (One way ANOVA)); and (FIG. 3F) images and a graph showing that MB5 treatment reduced both invasion (bottom left) as well as growth (bottom right) of spheroids generated from PC3 cells.

Images of Same Spheroid at Day 1 and Day 7 are Shown on Top Panels.

FIG. 4 includes graphs and a diagram showing a pharmacokinetic (PK) study of MB5 in a mouse model;

FIGS. 5A-5B include diagrams and graphs showing a comparison of two parent compounds MB5 and MB7, including diagrams and graphs showing: (FIG. 5A) the MB5 structure and its effect on reducing ouabain induced ERK activation in LLC-PK1 cells, n=3; and (FIG. 5B) the MB7 structure and its effect on reducing ouabain-induced ERK activation in LLC-PK1 cells.

FIGS. 6A-6G include images and graphs showing the effects of MB5 derivatives, including: (FIG. 6A-6D) images showing representative Western blots showing change in al NKA expression on treatment different concentrations of MB5 derivatives when treated up to 72 hours, where tubulin was used as loading control, and (FIG. 6E-6G) graphs showing quantitative analysis of spheroid invasion assay showing efficacy of MB5 derivatives in inhibiting spheroid invasion at a range of concentrations from 1-100 nM (*p<0.05 and **p<0.01 compared with 0 nM control of same compound, ##p<0.01 compared with 0 nM control of same compound, n.s.=not significant (One way ANOVA, multiple comparisons)).

FIGS. 7A-15C include graphs and images showing the results of ATPase activity assays to confirm the binding of MB5 derivatives to NKA, assays to measure the ability to increase the expression of α1 NKA and E-cadherin level, NKA biotinylation assays, and/or 3D cultures to test the anti-invasive and anti-growth potential of spheroids for various derivatives, including results of such assays using MIIRMB5 D1 (FIGS. 7A-7B), MIIRMB5 D3 (FIGS. 8A-8C), MIIRMB5 D4 (FIGS. 9A-9C), MIIRMB5 D5 (FIGS. 10A-10C), MIIRMB5 D6 (FIGS. 11A-11C), MIIRMB5 D7 (FIGS. 12A-12B), MIIRMB5 D13 (FIGS. 13A-13B), MIIRMB5 D14 (FIGS. 14A-14C), and MIIRMB5 D15 (FIGS. 15A-15C).

FIGS. 16A-16M includes images and graphs showing loss of α1 NKA in DU145 induces EMT and promotes invasion, including graphs and images showing: (FIG. 16A) generation cell subclones from DU145 and α1 NKA knockdown DU145 (KD) cells, where representative immunoblots for α1 and β1 NKA expression are shown for comparison; (FIG. 16B) quantitative analysis of α1 NKA expression (Western blot), relative to tubulin (**p<0.01, ***p<0.001 as indicated (N=4, One-way ANOVA)); (FIG. 16C) quantitative analysis of β1 NKA expression relative to tubulin (*p<0.05, n.s=not significant, as indicated (N=4)); (FIG. 16D) representative immunoblots for basal phospho and/or total forms of Src, FAK and Myc, where tubulin blot confirms equal loading (*p<0.05, **p<0.01 and ***p<0.001 relative to clone 5 (N=3-4, One-way ANOVA)); (FIG. 16E) representative phase contrast images of subclones 5, 4 and 2 (N=4, Scale bar=50 μm); (FIG. 16F) representative immunoblots (left) for epithelial (E-cadherin, ZO-1, ZO-2, occludin) and mesenchymal markers (SNAIL and ZEB1) in indicated subclones; (FIG. 16G) quantitative analyses (Western)*p<0.05 and **p<0.01 relative to clone 5 (N=4, one-way ANOVA); (FIG. 16H) qPCR analyses of EMT markers. *p<0.05 and **p<0.01 compared with sub-clone 5 (N=6, One-way ANOVA); (FIG. 16I) relative cell migration at 16 hours (Boyden chamber assay) **p<0.01 compared with sub-clone 5 (N=6, One-way ANOVA); (FIG. 16J) spheroid formation assay at day 7 (phase contrast images) (N=4, scale bar=50 μm); (FIG. 16K) spheroid invasion assay with representative images at different time points (N=8, scale bar=50 μm); (FIG. 16L) quantitative analysis of invasion (***p<0.0001 (N=8, Students t test); and (FIG. 16M) representative immunoblot for MMPs secreted by spheroids, where Ponceau stained nitrocellulose membrane is shown as loading control (N=3).

FIGS. 17A-17D include images and graphs showing α1 NKA endocytosis and EMT, including images and graphs showing: (FIG. 17A) α1 NKA endocytosis using cell surface biotinylation assay (**p<0.01 compared with DU145 (N=3, One-way ANOVA)); (FIG. 17B) representative confocal images of α1 NKA cellular distribution, where DU145 and PC3 are imaged at 3× exposure to C4-2 (Scale bar=50 μm); and (FIG. 17C) and (FIG. 17D) representative immunoblots showing α1 NKA, epithelial marker and mesenchymal marker expression in common PCa cell lines (top) with quantitative analyses (bottom) (* p<0.05 and **p<0.01 relative to C4-2 (N=4, Two-way ANOVA)).

FIGS. 18A-18F include images and graphs showing genetic rescue of α1 NKA counters tumor growth, including images and graphs showing: (FIG. 18A) representative immunoblot showing rat α1 NKA expression (anti-NASE antibody recognizes only rat al polypeptide) in rescued cells and parental DU145, where the bottom panel shows total α1 NKA expression (α6f antibody recognizes both human and rat NKA α1 polypeptide) (N=3); (FIG. 18B) a MTT assay showing effect of ouabain on cell viability of DU145, KD and rat α1 rescued cells. (N=5-6); (FIG. 18C) a cell proliferation assay, *p<0.05 as indicated (N=6, One-way ANOVA); (FIG. 18D) representative immunoblots showing Src activation (phospho-protein vs. total protein), total phosphotyrosine, Myc and tubulin expression (N=4); (FIG. 18E) quantitative analysis (Western) (*p<0.05 and **p<0.01 relative to DU145, N=3, One-way ANOVA); and (FIG. 18F) tumor weight from α1 KD and α1 rescued cell xenograft (*p<0.05, N=10, Students t test).

FIGS. 19A-19G include graphs and images showing the validation of MB5 as an inverse agonist of α1 NKA/Src signaling, including graphs and images showing: (FIG. 19A) al NKA endocytosis by biotinylation assay (**p<0.01 (N=3, One-way ANOVA); (FIG. 19B) confocal images of effect of ouabain and MB5 on phospho Src (activation) in LLC-PK1 (N=4); (FIG. 19C) confocal images of ERK activation/phosphorylation in LLC-PK1 cells (*p<0.05 and **p<0.01, n.s=not statistically significant, N=3, One-way ANOVA, images are at same scale); (FIG. 19D) effect of MB5 and ouabain treatment on ERK activation in LLC-PK1 cells (Western blot) (**p<0.01 compared with control and ##p<0.01 compared with only ouabain-treated group, N=3, One-way ANOVA); (FIG. 19E) effects of MB5 on ouabain-induced rat α1 NKA (YFP-tagged) endocytosis (Immunostaining) (###p<0.001 compared with control; **p<0.01 and ***p<0.001 compared with only ouabain treated cells (N=3, One-way ANOVA); (FIG. 19F) effect of MB5 on basal ERK phosphorylation in PY17 cells (*p<0.05 and **p<0.01 compared with control, N=3, One-way ANOVA); and (FIG. 19G) effect of MB5 treatment on α1 NKA endocytosis (biotinylation assay) in subclone 4 (**p<0.01 and ***p<0.001 compared with control, N=3, One-way ANOVA).

FIGS. 20A-20F include images and graphs showing MB5 inhibited spheroid growth and invasion by reversing EMT and Src/FAK signaling, including images and graphs showing: (FIG. 20A) representative immunoblots showing effect of 100 nM MB5 treatment on expression of EMT markers, Src/FAK activation and cell proliferation markers (Myc and PCNA) in DU145 derived subclones (loading control-tubulin, *p<0.05 and **p<0.01 as indicated, Students t test, N=3-4); (FIG. 20B) confocal images showing effect of 1 μM MB5 treatment on E-cadherin and occludin expression in subclone 2 after 16 hours (N=3, scale bar=50 μm); (FIG. 20C) representative images (top) and quantitative analyses (bottom) showing effect of MB5 treatment on invasion and growth of subclone 2 spheroids (**p<0.01 compared with 0 nM (N=4, One-way ANOVA); (FIG. 20D) representative immunoblots showing effect of MB5 treatment on MMPs secretion by subclone 2 spheroids (N=3, loading control=Ponceau stained nitrocellulose membrane); (FIG. 20E) Boyden chamber migration assay of subclone 2 cells pretreated with MB5, PP2 (Src inhibitor) or FAK inhibitor (DMSO=vehicle, n.s.=not significant, **p<0.01 compared with −DMSO treated group, N=6, One-way ANOVA); and (FIG. 20F) effect of MB5 treatment on growth of sub-clone 5 spheroids, representative images (top) and quantitative analyses (bottom) (*p<0.05 and **p<0.01 compared with 0 nM, N=4, One-way ANOVA).

FIGS. 21A-21E include images and graphs showing the effect of MB5 on PC3 and C4-2, including images and graphs showing: (FIG. 21A) the effect of MB5 treatment on PC3 spheroid invasion and growth, with representative images (top) and quantitative analyses (bottom) (*p<0.05 and **p<0.01 compared with 0 nM, N=6, One-way ANOVA), and with spheroid growth from day 1 to 7 (**p<0.01 compared with 0 nM, N=5, One-way ANOVA); (FIG. 21B) representative images of E-cadherin immunostaining in PC3 cells in ultralow attachment plate. (N=4, scale bar=50 μm); (FIG. 21C) representative immunoblots showing effect of MB5 on α1 NKA and Myc expression and Src activation in PC3 (*p<0.05 and **p<0.01 as indicated, N=3, Students t test); (FIG. 21D) representative blot of cytoplasmic and nuclear fractions on EMT markers in PC3 cells with MB5 treatment (N=3); (FIG. 21E) representative blot showing effect of 1 μM MB5 treatment on EMT phenotype in α1 KD cells derived from C4-2, *p<0.05 and **p<0.01 as indicated (N=3, Students t test).

FIGS. 22A-22D include images and graphs showing the effect of MB5 treatment on xenografted tumor growth in NOD/SCID mice, including images and graphs showing: (FIG. 22A) the effect of MB5 treatment (20 mg/kg/day) on tumor growth in NOD/SCOD mice xenografted with DU145 or α1 KD cells, with quantitative analysis of tumor weight (bottom) and representative images of tumors with/without MB5 treatment (top) (*p<0.05,***p<0.001 as indicated (Students' t test), N=10 mice per group); (FIG. 22B) tumor volume and weight of xenografted subclone 4 and 2 cells (***p<0.001 (One way ANOVA),*p<0.05 (Students' t test), N=10). (FIG. 22C) the effect of MB5 treatment (10 mg/kg/day) on xenografted tumor growth from aggressive sub-clones 4 and 2 (***p<0.001 and *p<0.05 as indicated (Students t test), N=10 tumors per group), with tumor weight (bottom) (*p<0.05 (Students t test)); (FIG. 22D) protein expression analyses of tumor lysates (from sub-clone 2) by Western blot (***p<0.001 and *p<0.05 as indicated (One-way ANOVA), N=4-6 tumors per group).

FIGS. 23A-23B include schematic diagrams showing a graphical abstract of molecular mechanism, including schematic diagrams showing: (FIG. 23A) the effect of tumor microenvironment on α1 NKA/Src receptor complex activation and its endocytosis leading to EMT; and (FIG. 23B) MB5 treatment blocks α1 NKA/Src receptor complex in inactive conformation and reverses EMT by stabilizing cell-cell attachment.

FIGS. 24A-24H include images and graphs showing: (FIG. 24A) representative immunoblots showing α1 NKA expression in tumor lysates and corresponding cell lines generated from xenograft-derived tumors; (FIG. 24B) representative immunostaining images for al NKA (N=4, scale bar=50 μm); (FIG. 24C) representative immunoblot for cell proliferation markers in subclones (*p<0.05, ***p<0.001, N=4, One-way ANOVA); (FIG. 24D) phase contrast images of C4-2 and α1 knockdown cells (KD) (N=3-4, scale bar=50 μm); (FIG. 24E) representative immunoblots showing EMT markers and loading control (tubulin) (*p<0.05 and **p<0.01 relative to C4-2, N=3, Students' t test); (FIG. 24F) qPCR analyses of mesenchymal markers (*p<0.05, **p<0.01 (N=6, One-way ANOVA); and (FIG. 24G) representative immunoblots for Src, FAK and ERK activation (phosphoprotein/total protein), *p<0.05 (N=3, Students' t test); (FIG. 24H) representative images of spheroid invasion assay (N=4, Scale bar-50 μm).

FIGS. 25A-25E include images and graphs showing: (FIG. 25A) the effect of MB5 treatment on Dopamine-induced ERK activation in LLC-PK1 cells (Western blot) (*p<0.05 and **p<0.01, n.s=not significant, N=4, One-way ANOVA); (FIG. 25B) the effect of MB5 treatment on EGF-induced ERK activation in LLC-PK1 cells (Western blot) (***p<0.001, n.s=not significant, N=4, One-way ANOVA); (FIG. 25C) confocal images of ERK activation (phospho ERK) by Dopamine with or without MB5 (***p<0.001, n.s=not significant, N=3, One-way ANOVA); (FIG. 25D) confocal images of ERK activation by EGF with or without MB5 (**p<0.01, n.s=not significant, N=3, One-way ANOVA); and (FIG. 25E) the effect of MB5 on basal ERK activation in Src binding mutant Y260A and A425P cells (n.s=not significant compared with control, N=3, One-way ANOVA).

FIGS. 26A-26E include graphs and images showing: (FIG. 26A) the effect of PP2 treatment (24 hours) on E-cadherin expression in sub-clone 2 (tubulin-loading control, N=3, Students t test); (FIG. 26B) the effect of MB5 treatment on Src activation (phospho Src vs. Src) in immunoprecipitated Src protein from PC3 cells (N=3, Students t test); (FIG. 26C) quantitative analysis of C4-2 spheroid growth Day 1-7 with or without MB5 (**p<0.01 compared with untreated spheroid on same day, N=4, One-way ANOVA); (FIG. 26D) comparison of body weight between DMSO (vehicle) or MB5 (20 mg/kg) treated NOD/SCID mice (n.s=not significant, N=10, Students t test); and (FIG. 26E) a table showing summarized effect of MB5 treatment on tumor growth of different DU145 derived cells in xenografted mice model.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

The present application can “comprise” (open ended), “consist of” (closed ended), or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments±20%, in some embodiments ±10%, in some embodiments±5%, in some embodiments±1%, in some embodiments±0.5%, and in some embodiments±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is based, at least in part, on the discovery that significant loss of α1 NKA expression grants cancer cells an ability to undergo metastasis and that a resolution to that issue is to reduce the endocytosis of α1 NKA in cancer cells. In this regard, the presently-disclosed subject matter includes a new class of α1 NKA ligands that are capable of decreasing the endocytosis of α1 NKA and restoring the expression of α1 NKA in the plasma membrane. In particular, and without wishing to be bound by any particular theory or mechanism, it is believed that these new compounds are capable of reversing the EMT process and reducing tumor growth and invasion in cancer, including, in some embodiments, advanced prostate cancer. Moreover, these ligands are believed to have pharmacokinetic properties better than currently-available compounds targeting the α1 NKA as the ligands of the presently-disclosed subject matter have substituted phenolic groups and other chemical structures that improve their ability to be used as therapeutic agents.

In some embodiments, a compound is provided that comprises a α1 NKA ligand. In some embodiments, a compound is provided that comprises the following formula (I):

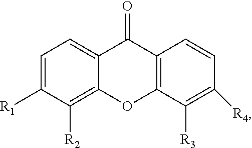

where R₁, R₂, R₃, and R₄ are independently selected from a hydrogen atom, a hydroxyl, an amine, a sulfonic acid, a thiol, a fluorine atom, or a phosphate group, or where R₁ and R₂ are a hydrogen and R₃ and R₄ are combined to produce a heterocyclic group including one or more nitrogen atoms.

With regard to the various substituent groups of the presently-described compounds, as used herein, the term “hydroxyl” refers to an —OH group, while the term “amine” is used to refer to a functional group consisting of a nitrogen atom with three single bonds to either hydrogen atoms or alkyl groups. In some embodiments, a primary amine can thus be defined as a nitrogen atom bonded to two hydrogen atoms and one other group (R—NH2), a secondary amine can be defined as a nitrogen atom bonded to one hydrogen atom and two other groups (R—NH—R), and a tertiary amine is defined as a nitrogen atom bonded to three other groups (R3N).

The term “sulfonic acid” (or sulphonic acid or sulfo group) is used herein to refer to a member of the class of organosulfur compounds with the general formula —S(═O)2-OH. The term “thiol” is used to refer to a sulfur atom bonded to a hydrogen atom (—SH), while the term “phosphate group” refers to a substituent group including one atom of phosphorus covalently bound to four oxygen residues, two of which may be expressed as a hydroxyl group. The phrase “heterocyclic group including one or more nitrogen atoms” is used to refer to a substituent group containing a saturated or wholly or partially unsaturated 4-10 membered ring containing one or more nitrogen atoms.

In some embodiments of the presently-disclosed subject matter, a compound of formula (I) is provided having the following formula (II):

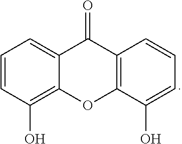 [SMILES: O=c1c2cccc(O)c2oc2c(O)cccc12]

As another example of a compound of formula (I), in some embodiments, a compound of formula (I) is provided having the following formula (III):

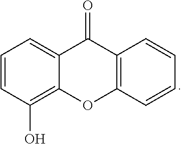 [SMILES: O=c1c2ccccc2oc2c(O)cccc12]

As another example, in some embodiments, a compound of formula (I) is provided having the following formula (IV):

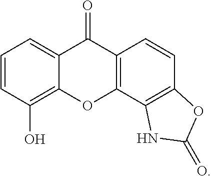 [SMILES: O=c1[nH]c2c(ccc3c(=O)c4cccc(O)c4oc32)o1]

As another example, in some embodiments, a compound of formula (I) is provided having the following formula (V):

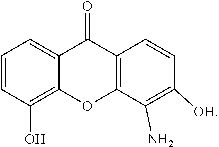 [SMILES: Nc1c(O)ccc2c(=O)c3cccc(O)c3oc12]

As another example, in some embodiments, a compound of formula (I) is provided having the following formula (VI):

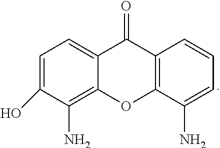 [SMILES: Nc1cccc2c(=O)c3ccc(O)c(N)c3oc12]

As another example, in some embodiments, a compound of formula (I) is provided having the following formula (VII):

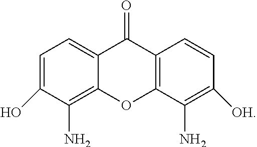 [SMILES: Nc1c(O)ccc2c(=O)c3ccc(O)c(N)c3oc12]

As another example, in some embodiments, a compound of formula (I) is provided having the following formula (VIII):

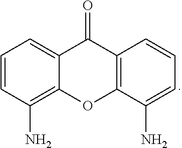 [SMILES: Nc1cccc2c(=O)c3cccc(N)c3oc12]

As another example, in some embodiments, a compound of formula (I) is provided having the following formula (IX):

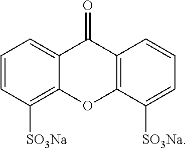

As another example, in some embodiments, a compound of formula (I) is provided having the following formula (X):

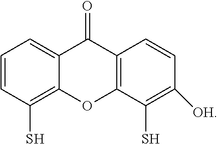 [SMILES: O=c1c2cccc(S)c2oc2c(S)c(O)ccc12]

As another example, in some embodiments, a compound of formula (I) is provided having the following formula (XI):

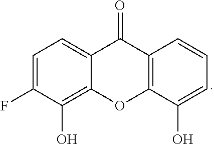 [SMILES: O=c1c2cccc(O)c2oc2c(O)c(F)ccc12]

As another example, in some embodiments, a compound of formula (I) is provided having the following formula (XII):

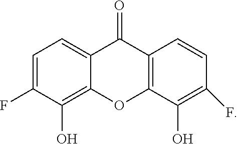 [SMILES: O=c1c2ccc(F)c(O)c2oc2c(O)c(F)ccc12]

As another example, in some embodiments, a compound of formula (I) is provided having the following formula (XIII):

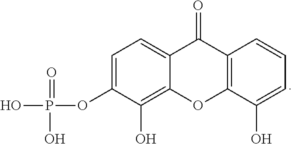 [SMILES: O=c1c2cccc(O)c2oc2c(O)c(OP(=O)(O)O)ccc12]

In some embodiments of the Na/K-ATPase ligands described herein, a compound is provided the formula selected from the group consisting of:

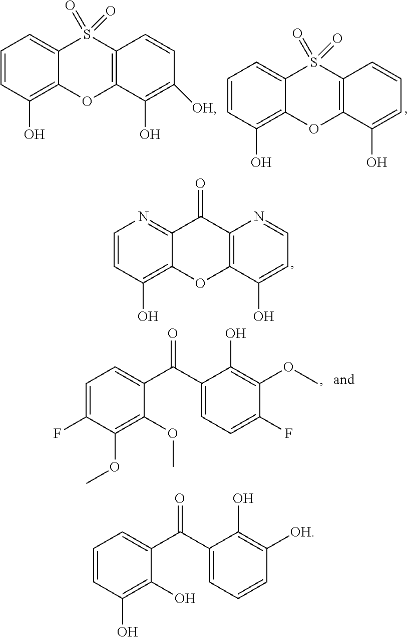

In other embodiments of the Na/K-ATPase ligands described herein, a compound is provided having a formula selected from:

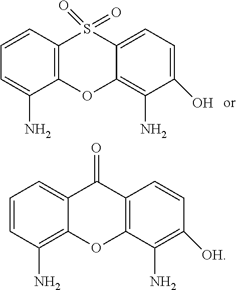 [SMILES: Nc1cccc2c1Oc1c(ccc(O)c1N)S2(=O)=O ; Nc1cccc2c(=O)c3ccc(O)c(N)c3oc12]

Further provided, in some embodiments of the presently-disclosed subject matter, are pharmaceutical compositions that include the compounds (e.g., the α1 Na/K-ATPase ligands) described herein and a pharmaceutically-acceptable vehicle, carrier, or excipient. Indeed, when referring to certain embodiments herein, the terms “α1 Na/K-ATPase ligands” and/or “compound” and the like may or may not be used to refer to a pharmaceutical composition that includes the α1 Na/K-ATPase ligands.

The term “pharmaceutically-acceptable carrier” as used herein refers to sterile aqueous or nonaqueous solutions, dispersions, suspensions, or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These compositions can also contain adjuvants, such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid, and the like. It can also be desirable to include isotonic agents such as sugars, sodium chloride and the like.

Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of compound to biodegradable polymer and the nature of the particular biodegradable polymer employed, the rate of compound release can be controlled. Depot injectable formulations can also be prepared by entrapping the compound in liposomes or microemulsions, which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

Suitable formulations can further include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents.

The compositions can also take forms such as suspensions, solutions, or emulsions in oily or aqueous vehicles, and can contain formulatory agents such as suspending, stabilizing and/or dispersing agents. Alternatively, the compounds can be in powder form for constitution with a suitable vehicle, e.g., sterile pyrogen-free water, before use.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets can be coated by methods known in the art.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically-acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration, the compositions can take the form of tablets or lozenges formulated in a conventional manner.

The compositions can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). The compounds can also be formulated in rectal compositions, creams or lotions, or transdermal patches.

Still further provided, in some embodiments of the presently-disclosed subject matter, are methods for treating a cancer. In some embodiments, a method for treating a cancer is provided that comprises administering to a subject in need thereof an effective amount of a composition of the presently-disclosed subject matter comprising a α1 Na/K-ATPase ligand described herein (e.g., a compound of formula (I) above).

As used herein, the terms “treating” or “treatment” relate to any treatment of a cancer including, but not limited to, therapeutic treatment and prophylactic treatment of a cancer. With regard to therapeutic treatment of a cancer, the terms “treating” or “treatment” include, but are not limited to, inhibiting the progression of a cancer, arresting the development of a cancer, reducing the severity of a cancer, ameliorating or relieving one or more symptoms associated with a cancer, and causing a regression of a cancer or one or more symptoms associated with a cancer.

As noted herein above, the terms “treating” or “treatment,” further include the prophylactic treatment of a cancer including, but not limited to, any action that occurs before the development of a cancer. It is understood that the degree of prophylaxis need not be absolute (e.g. the complete prophylaxis of a cancer such that the subject does not develop a cancer at all), and that intermediate levels of prophylaxis, such as increasing the time required for at least one symptom resulting from a cancer to develop, reducing the severity or spread of a cancer in a subject, or reducing the time that at least one adverse health effect of a cancer is present within a subject, are all examples of prophylactic treatment of a cancer.

As further non-limiting examples of the treatment of a cancer by a composition described herein, treating a cancer can include, but is not limited to, killing cancer cells, inhibiting the development of cancer cells, inducing apoptosis in cancer cells, reducing the growth rate of cancer cells, reducing the incidence or number of metastases, reducing tumor size, inhibiting tumor growth, reducing the available blood supply to a tumor or cancer cells, promoting an immune response against a tumor or cancer cells, reducing or inhibiting the initiation or progression of a cancer, or increasing the lifespan of a subject with a cancer.

With respect to the cancer treated in accordance with the presently-disclosed subject matter, the term “cancer” is used herein to refer to all types of cancer or neoplasm or malignant tumors found in animals, including leukemias, carcinomas, melanoma, and sarcomas. Examples of cancers are cancer of the brain, bladder, breast, cervix, colon, head and neck, kidney, lung, non-small cell lung, mesothelioma, ovary, prostate, sarcoma, stomach, uterus and Medulloblastoma. In some embodiments, the cancer is prostate cancer. In some embodiments, the cancer is a metastatic cancer.

By “leukemia” is meant broadly progressive, malignant diseases of the blood-forming organs and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “sarcoma” generally refers to a tumor which is made up of a substance like the embryonic connective tissue and is generally composed of closely packed cells embedded in a fibrillar or homogeneous substance. Sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

Additional cancers include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer. In some embodiments, the cancer is prostate cancer.

In some embodiments of the presently-disclosed methods of treating a cancer, the cancer can be a primary cancer or a secondary cancer. As used herein, the term “primary cancer” is meant to refer to an original tumor or cancer cell in a subject. Such primary cancers are usually named for the part of the body in which the primary cancer originates. Furthermore, a “secondary cancer” is used herein to refer to a cancer which has spread, or metastasized, from an initial site (i.e. a primary cancer site) to another site in the body of a subject, a cancer which represents a residual primary cancer, or a cancer that has originated from treatment with an antineoplastic agent(s) or radiation or both. In this regard, the term “secondary cancer” is thus not limited to any one particular type of cancer, including the type of primary cancer from which it derived. In some embodiments of the presently-disclosed subject matter, a method of preventing or treating a cancer is further provided where the subject is at risk of developing a secondary cancer.

Suitable methods for administering a therapeutic composition in accordance with the methods of the presently-disclosed subject matter include, but are not limited to, systemic administration, parenteral administration (including intravascular, intramuscular, and/or intraarterial administration), oral delivery, buccal delivery, rectal delivery, subcutaneous administration, intraperitoneal administration, inhalation, intratracheal installation, surgical implantation, transdermal delivery, local injection, intranasal delivery, and hyper-velocity injection/bombardment. Where applicable, continuous infusion can enhance drug accumulation at a target site (see, e.g., U.S. Pat. No. 6,180,082). In some embodiments, the administration of the composition is via oral administration, transdermal administration, administration by inhalation, nasal administration, topical administration, intraaural administration, rectal administration, intravenous administration, intramuscular administration, subcutaneous administration, intravitreous administration, subconjunctival administration, intracameral administration, intraocular administration or combinations thereof.

Regardless of the route of administration, the compositions of the presently-disclosed subject matter are typically administered in amount effective to achieve the desired response. As such, the term “effective amount” is used herein to refer to an amount of the therapeutic composition (e.g., an α1 Na/K-ATPase ligands and a pharmaceutically vehicle, carrier, or excipient) sufficient to produce a measurable biological response (e.g., a decrease in metastasis). Actual dosage levels of active ingredients in a therapeutic composition of the present invention can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, New Jersey; Goodman et al., (1996) Goodman & Gilman's the Pharmacological Basis of Therapeutics, 9th ed. McGraw-Hill Health Professions Division, New York; Ebadi, (1998) CRC Desk Reference of Clinical Pharmacology. CRC Press, Boca Raton, Fla.; Katzung, (2001) Basic & Clinical Pharmacology, 8th ed. Lange Medical Books/McGraw-Hill Medical Pub. Division, New York; Remington et al., (1975) Remington's Pharmaceutical Sciences, 15th ed. Mack Pub. Co., Easton, Pa.; and Speight et al., (1997) Avery's Drug Treatment: A Guide to the Properties, Choice, Therapeutic Use and Economic Value of Drugs in Disease Management, 4th ed. Adis International, Auckland/Philadelphia; Duch et al., (1998) Toxicol. Lett. 100-101:255-263.

In some embodiments of the presently-disclosed subject matter, the compound or compositions described herein are administered in an amount sufficient to reduce an endocytosis of an α1 Na/K-ATPase in a cancer cell, such as a cancer cell present within a subject.

As used herein, the term “subject” includes both human and animal subjects. Thus, veterinary therapeutic uses are provided in accordance with the presently disclosed subject matter. As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The practice of the presently-disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The presently-disclosed subject matter is further illustrated by the following specific but non-limiting examples. The examples may include compilations of data that are representative of data gathered at various times during the course of development and experimentation related to the presently-disclosed subject matter.

### EXAMPLES

Examples 1-4 describe the development of a screening assay to identify α1 NKA ligands that work differently from cardiotonic steroids. An analysis was undertaken to identify chemical structures that block cardiotonic steroid (CTS)-induced signal transduction via the al NKA/Src complex, and consequently reduce the endocytosis of α1 NKA in cancer cells. That analysis was then followed by a functional analyses both in vitro and in vivo to test the efficacy of the new compounds for their ability to reduce EMT and metastatic potential of cancer cells and to decrease the growth of tumor xenograft.

As described below, a new class of α1 NKA ligands was identified that were able to decrease the endocytosis of α1 NKA and restore the expression of α1 NKA in the plasma membrane. The parent compound named MB5 was then further used to show that MB5 treatment can reverse the EMT process and stop tumor growth and invasion in advanced prostate cancer. Moreover, it was observed that the derivatives of MB5 exhibited improved structural properties that had either better or similar efficacy and potency as MB5. Because MB5 structure contains 3 phenolic groups, which made its pharmacokinetic properties less suitable for development as a drug candidate, the newly developed derivatives also had better pharmacokinetic properties, as the phenolic groups were substituted with other structures that improve their ability to be utilized as therapeutic agents.

### Example 1—MB5, the Parent Compound, Increases α1 NKA Expression in Cells by Preventing its Endocytosis

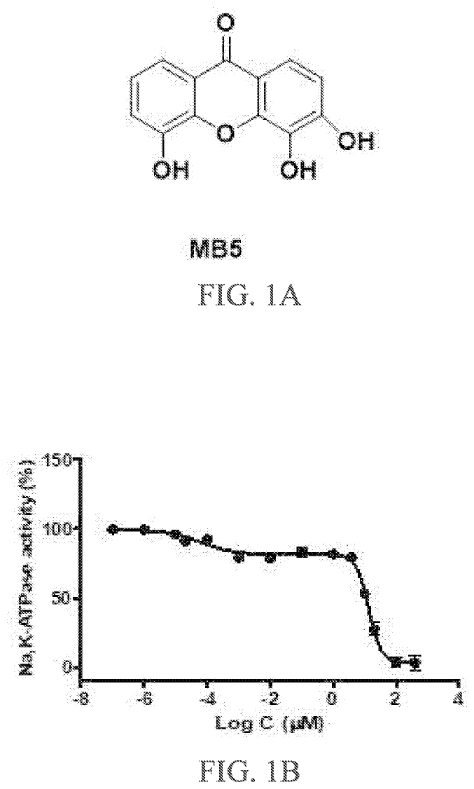

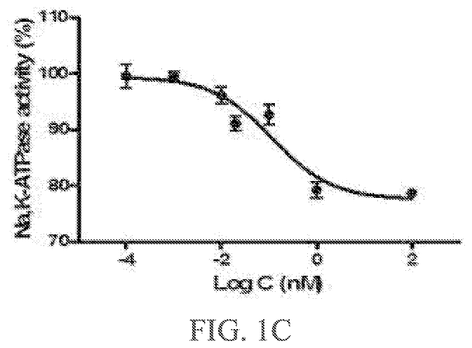

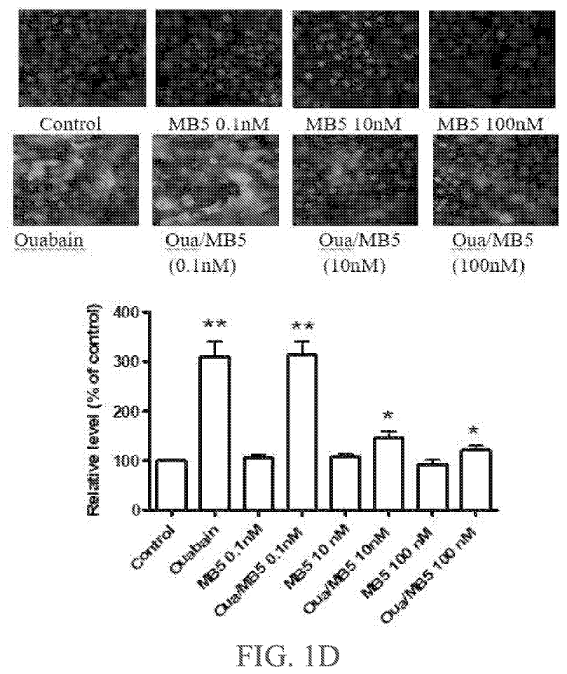

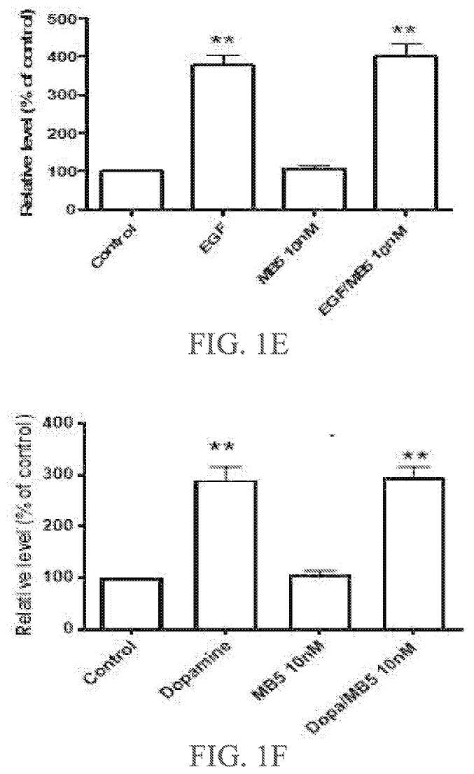

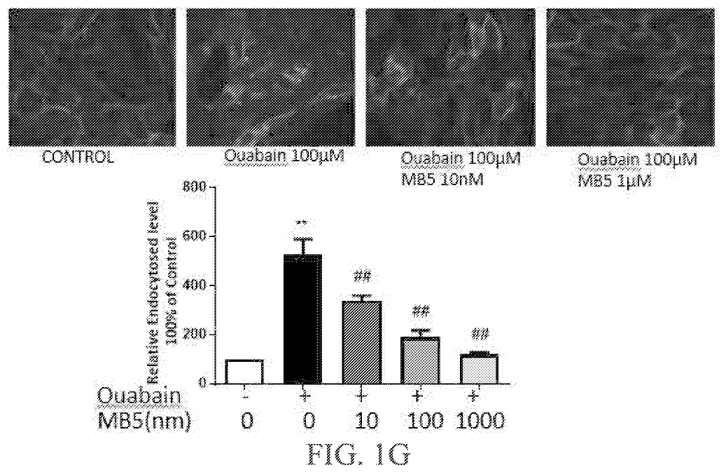

FIG. 1A shows the chemical structure of MB5. FIGS. 1B-1C shows that MB5 is a NKA ligand, as it was able to inhibit activity of purified pig kidney NKA with an IC₅₀ value of about 10 μM. Interestingly MB5 inhibition of NKA activity exhibited a biphasic curve, with an inhibitory concentration of about 1 nM to 100 nM (˜25% inhibition) and ˜10 μM (˜50% inhibition). It is appreciated that ouabain induces Src/ERK activation (increased protein phosphorylation) of LLC-PK1 cells by binding to α1 NKA and thereby initiates a signaling cascade which results in the endocytosis of the α1 NKA to terminate the signaling. As shown in FIG. 1D, 10 minutes of MB5 pretreatment of LLC-PK1 cells abolished ouabain-induced ERK activation as depicted by immunostaining of phosphorylated ERK and imaged using confocal microscopy. Furthermore, MB5 did not inhibit EGF or dopamine induced ERK activation (FIGS. 1E-1F) indicating that it is a specific ligand of NKA. Moreover, MB5 was able to inhibit ouabain-induced α1 NKA endocytosis in YFP-al-TCN cells, a cell line that expresses YFP-tagged α1 NKA (FIG. 1G). Thus this dataset confirms that MB5 works by acting as an inverse agonist of α1 NKA/Src receptor complex.

### Example 2—MB5 Prevents Tumor Invasion and Growth of Prostate Cancer Cells

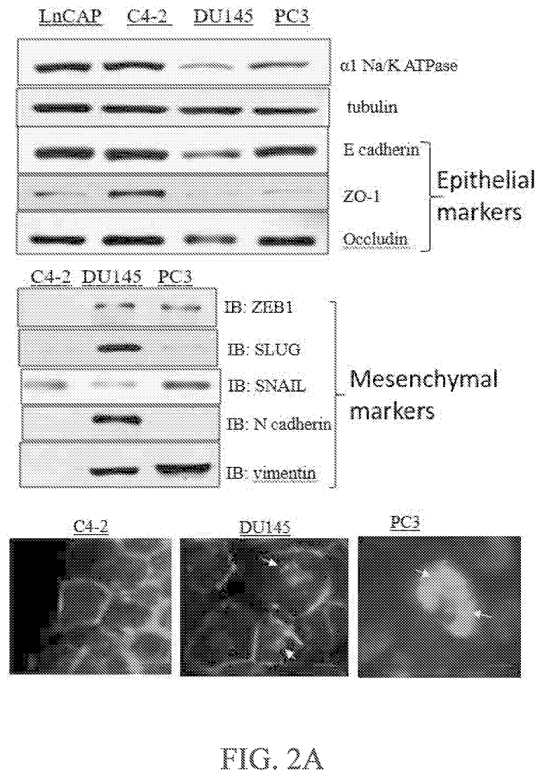

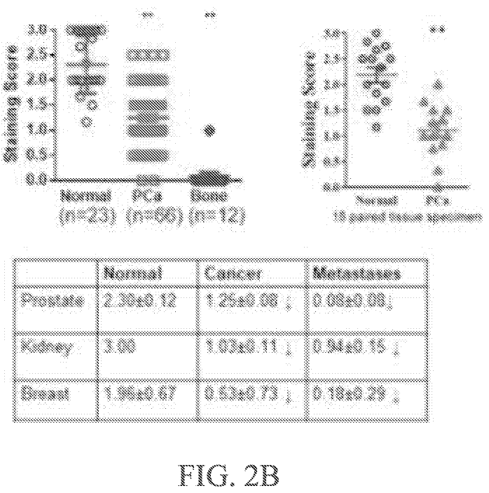

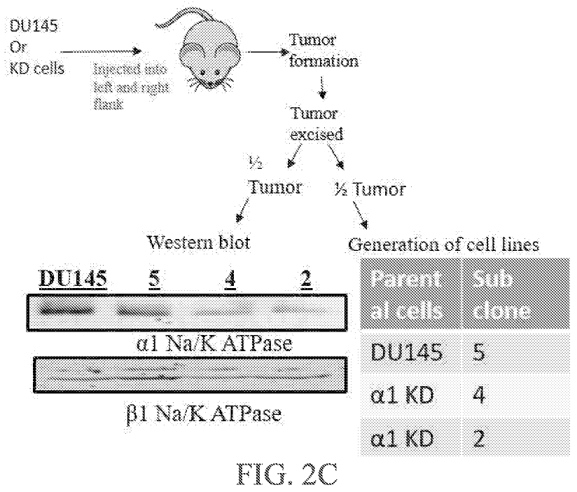

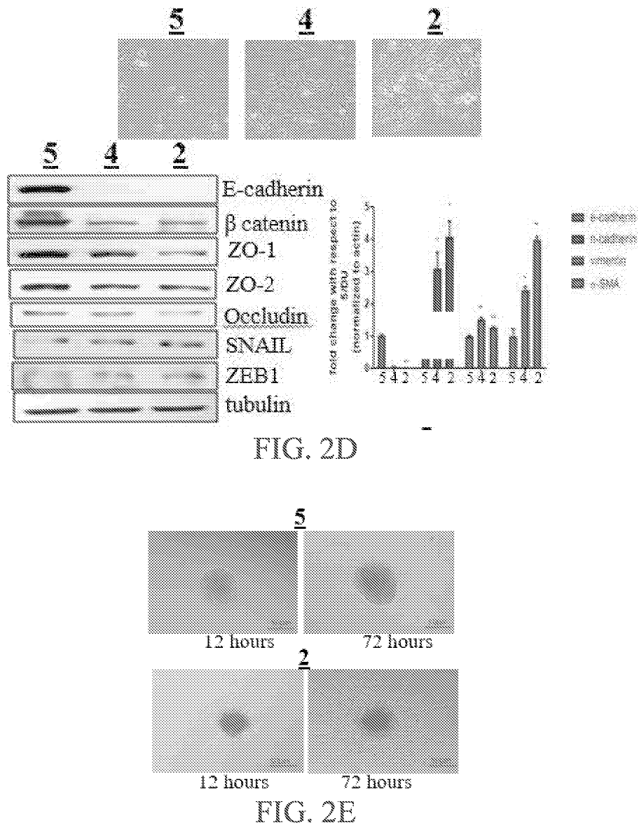

A comparison of α1 NKA expression among three widely used aggressive prostate cancer cell lines showed that loss of α1 NKA expression because of increased endocytosis, was associated with the EMT phenotype (FIG. 2A). Also, α1 NKA expression was significantly reduced in primary tumor and barely detectable in metastatic sites (FIG. 2B). It was therefore hypothesized that the loss of α1 NKA enhances the metastatic potential of prostate cancer cells. As shown in FIG. 2C, by xenograft transplantation, three clonal population of cells were generated which expressed only 10%, 20% and 70% of α1 NKA relative to parental DU145 cell line. FIG. 2D shows that the loss of α1 NKA expression resulted in EMT as measured by loss of epithelial markers (E-cadherin, ZO-1, ZO-2 and occludin), but upregulation of mesenchymal markers (Zeb1, SNAIL, Vimentin and N-cadherin). In 3D culture, clone 2 which expressed the least amount of α1 NKA (about 10% of parental DU145 cells) invaded into the matrix in only 12 hours. This was in sharp contrast to clone 5 that expressed about 70% α1 NKA and did not invade into the matrix even after 3 days (FIG. 2E).

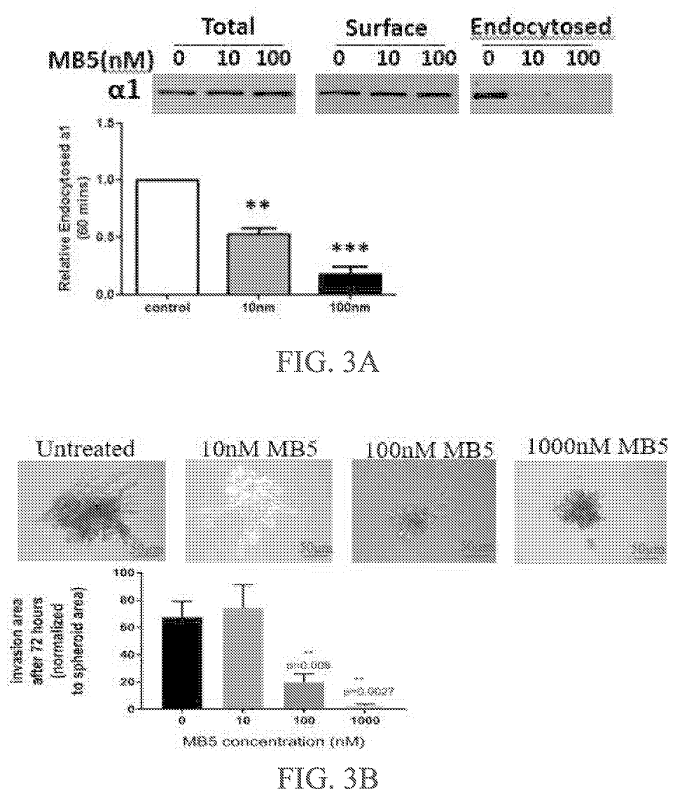

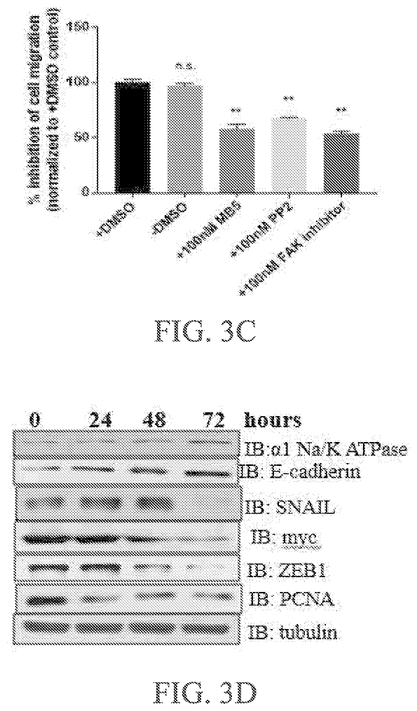

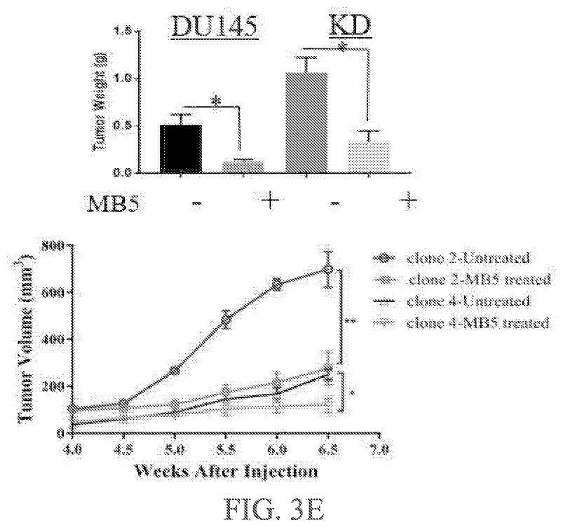

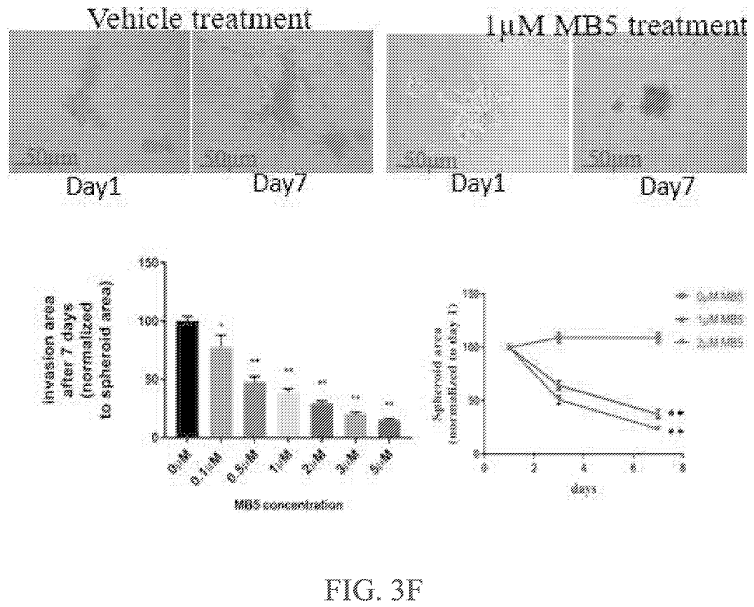

It was therefore tested whether MB5 could reduce the invasive potential of prostate cancer cells by preventing α1 NKA endocytosis. FIG. 3A shows that MB5 significantly reduced al endocytosis of xenograft derived clone 4 (˜20% expression of α1 NKA). In 3D culture, MB5 treatment abolished the invasion of clone 2 spheroids (FIG. 3B). This was further confirmed by Boyden chamber assay which showed that MB5 treatment inhibited migration of cancer cells by about 50% (FIG. 3C). Finally, Western blot analysis confirmed that MB5 worked by increasing al NKA as well as E-cadherin level in prostate cancer cells which reversed the EMT phenotype by downregulating the expression of mesenchymal markers—ZEB1, SNAIL as well as c-Myc (FIG. 3D). In vivo studies showed that MB5 treatment significantly reduced tumor growth from all tested cell lines when xenografted into NOD/SCID mice (FIG. 3E). MB5 also reduced spheroid invasion and growth of PC3 cells (FIG. 3F), indicating that it is applicable in all types of prostate cancer cells.

### Example 3—Design of Novel MB5 Derivative Compounds

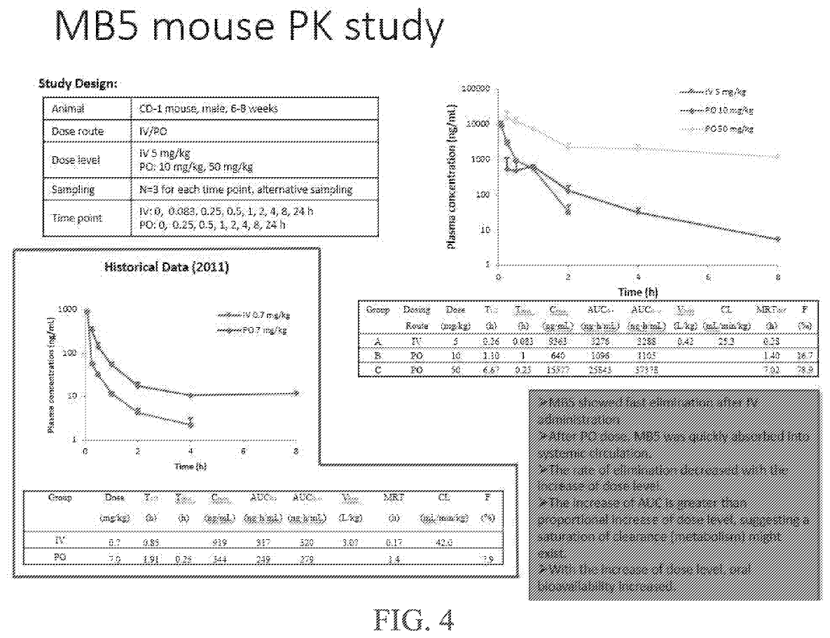

As shown in FIG. 1A, MB5 contains 3 phenolic groups which are problematic in terms of pharmacokinetic properties, making it a less ideal drug candidate (FIG. 4). On the other hand, it is quite non-toxic, and the systemic exposure level is proportional to dose level in the range of 50-200 mg/kg after oral administration. No detectable toxic effects were observed after more than 1 month of administration. In addition, MB5 showed good plasma stability and was not metabolized significantly by P450 enzymes. However, it was quickly removed by secondary metabolism (most likely by UGT enzymes).

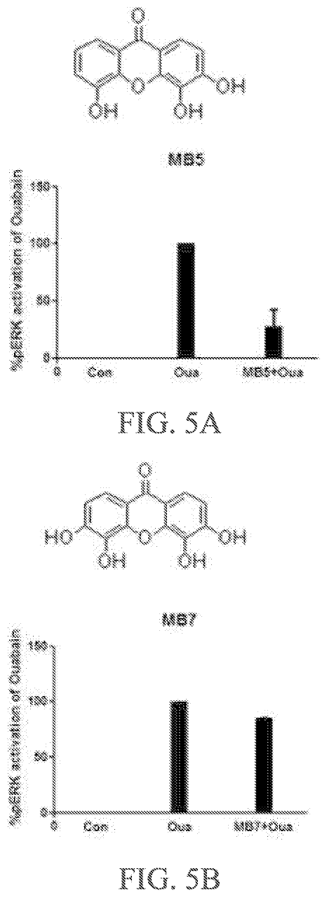

Given the shortcomings of MB5, a series of MB5 derivatives were designed as listed in Table 1 and synthesized using methods know to those skilled in the art to achieve the following goals. First, it was desirable to find novel structures that improved the efficacy; second, to improve potency, and third to improve the ability of the MB5 to be used as a drug by reducing secondary metabolism of the compounds. For example, a comparison between MB5 and MB7 (FIGS. 5A-5B) showed that elimination of one phenolic group (present in MB7) improved the efficacy of the compound significantly. MB5 which lacks one phenolic group shows better efficacy to inhibit ouabain-induced ERK activation than MB7.

As shown in Table 1, the MB5 derivatives were classified into four groups according to the type of their structural modification. Group 1 constitutes derivatives in which the number or the position of 3 phenolic groups have been changed, to assess which of these groups are most effective as inverse NKA/Src agonists. Group 2 consists of MB5 derivatives in which the phenolic groups have been substituted with other groups, to test whether these substitutions can improve the efficacy and potency, as well as pharmacokinetic properties. Group 3 contains derivatives where the ring structure has been altered to find substitutions, that are better or equal to the xanthone ring. Group 4 contains derivatives where the ring structure is broken. All together, these modifications provide for an assessment of the structure-activity relationship (SARS) of the compounds.

TABLE 1
MB5 Derivatives

| | |
|---|---|
| Compound | Structure |

Group I

| | |
|---|---|
| MIIRMB5-D1 | 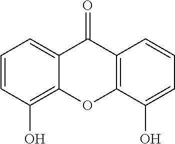 [SMILES: O=c1c2cccc(O)c2oc2c(O)cccc12] |
| MIIRMB5-D2 | 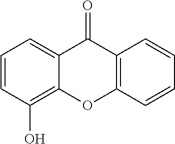 [SMILES: O=c1c2ccccc2oc2c(O)cccc12] |

Group 2

| | |
|---|---|
| MIIRMB5-D3 | 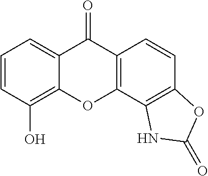 [SMILES: O=c1[nH]c2c(ccc3c(=O)c4cccc(O)c4oc32)o1] |
| MIIRMB5-D4 | 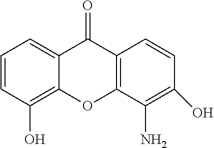 [SMILES: Nc1c(O)ccc2c(=O)c3cccc(O)c3oc12] |
| MIIRMB5-D5 | 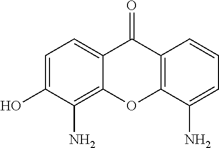 [SMILES: Nc1cccc2c(=O)c3ccc(O)c(N)c3oc12] |
| MIIRMB5-D6 | 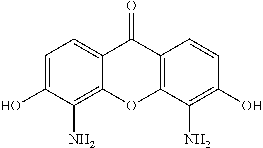 [SMILES: Nc1c(O)ccc2c(=O)c3ccc(O)c(N)c3oc12] |
| MIIRMB5-D7 | 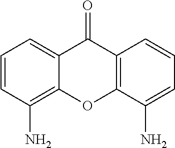 [SMILES: Nc1cccc2c(=O)c3cccc(N)c3oc12] |
| MIIRMB5-D8 | 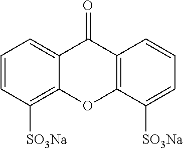 |
| MIIRMB5-D9 | 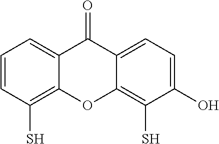 [SMILES: O=c1c2cccc(S)c2oc2c(S)c(O)ccc12] |
| MIIRMB5-D10 | 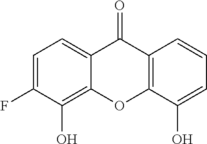 [SMILES: O=c1c2cccc(O)c2oc2c(O)c(F)ccc12] |
| MIIRMB5-D11 | 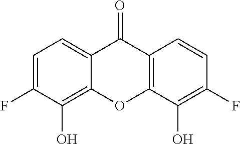 [SMILES: O=c1c2ccc(F)c(O)c2oc2c(O)c(F)ccc12] |
| MIIRMB5-D12 | 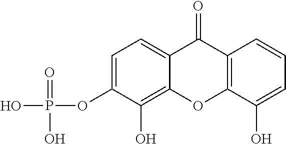 [SMILES: O=c1c2cccc(O)c2oc2c(O)c(OP(=O)(O)O)ccc12] |

Group 3

| | |
|---|---|
| MIIRMB5-D13 | 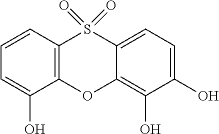 [SMILES: O=S1(=O)c2cccc(O)c2Oc2c1ccc(O)c2O] |
| MIIRMB5-D14 | 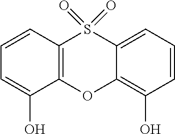 [SMILES: O=S1(=O)c2cccc(O)c2Oc2c(O)cccc21] |
| MIIRMB5-D15 | 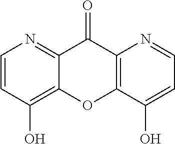 [SMILES: O=c1c2nccc(O)c2oc2c(O)ccnc12] |

Group 4

| | |
|---|---|
| MIIRMB5-D16 | 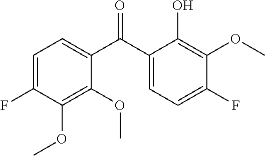 [SMILES: COc1c(F)ccc(C(=O)c2ccc(F)c(OC)c2OC)c1O] |
| MIIRMB5-D17 | 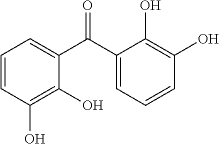 [SMILES: O=C(c1cccc(O)c1O)c1cccc(O)c1O] |

### Example 4—Development of Assay for Identifying Anti-Invasion and Anti-Tumor Growth Properties of MB5 Derivatives

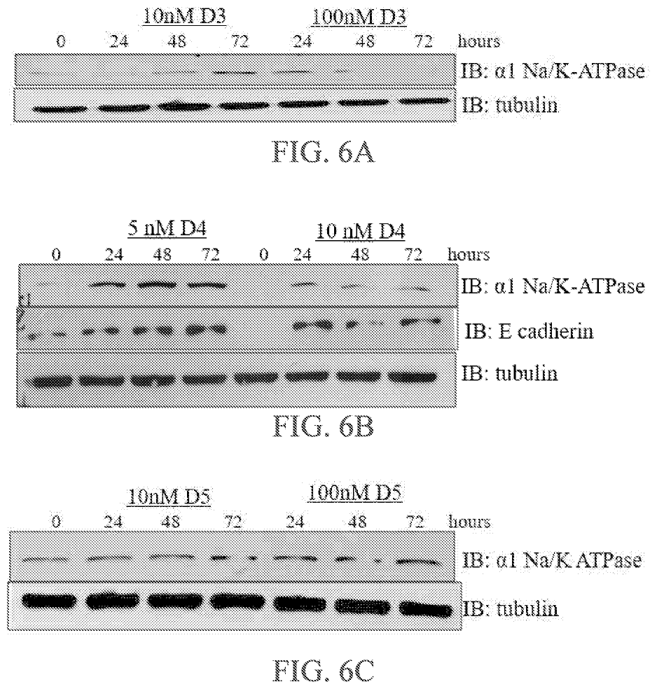

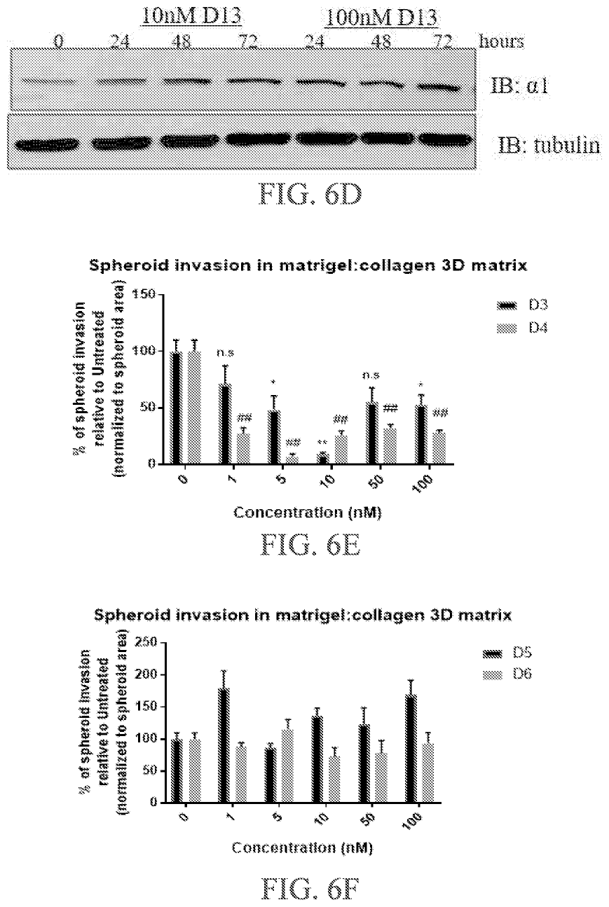

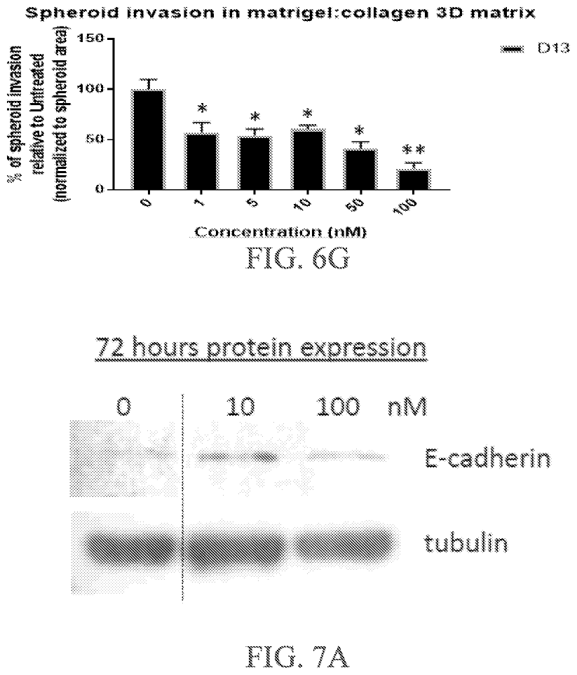

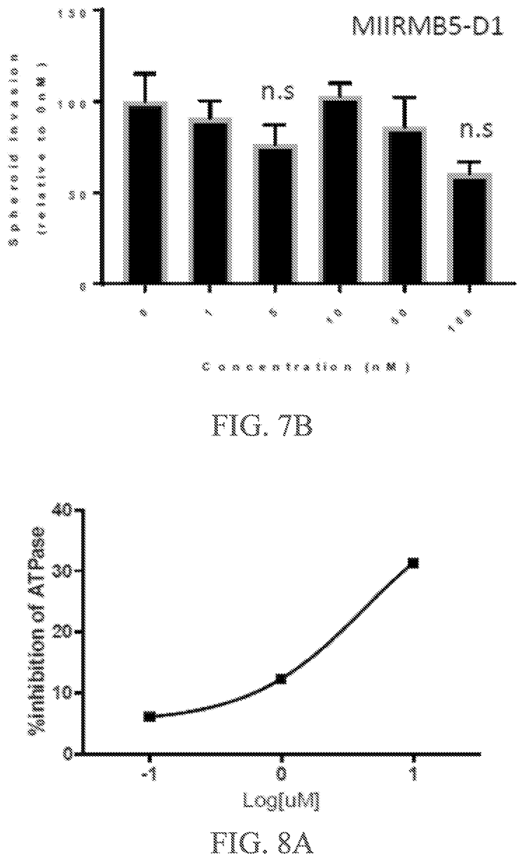

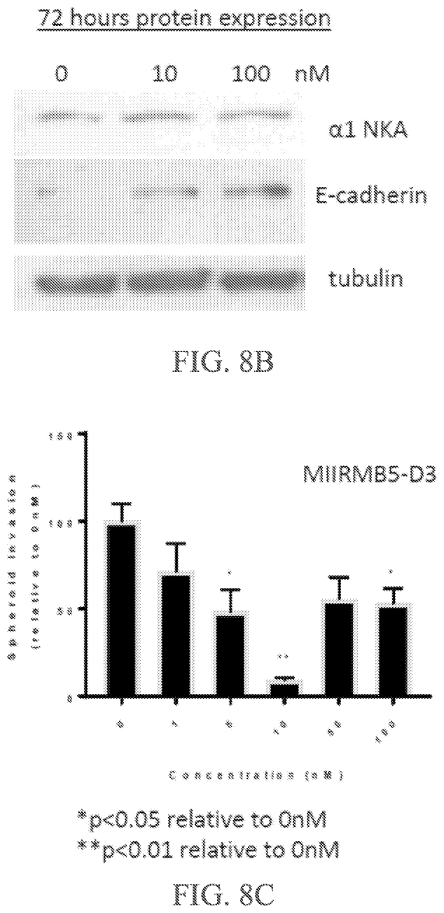

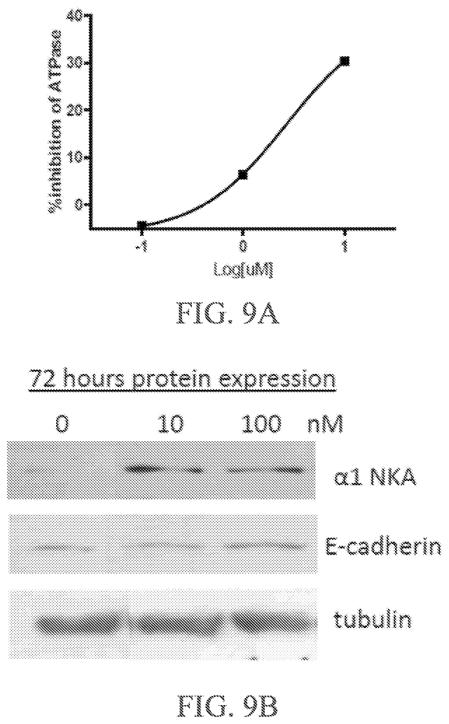

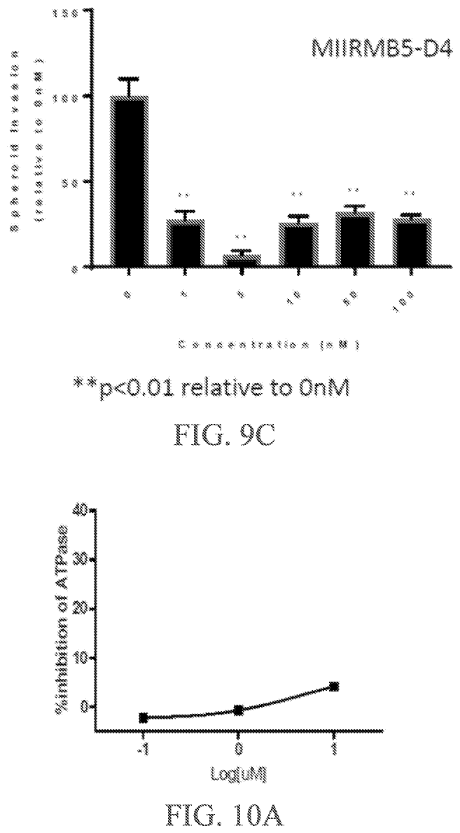

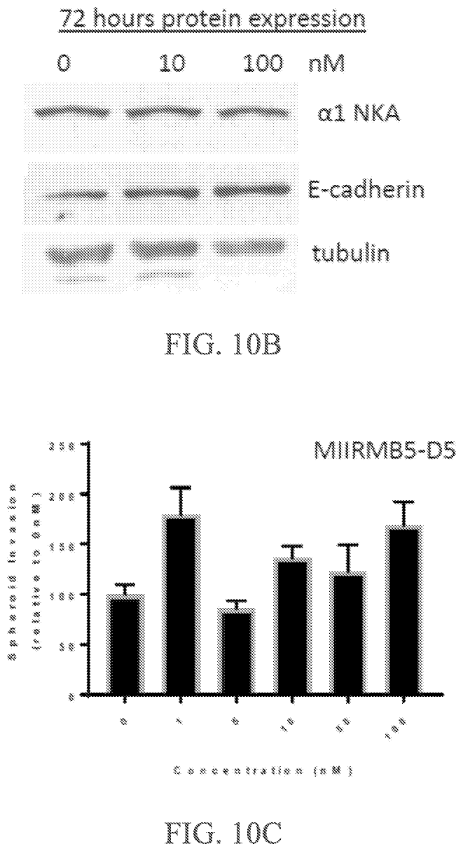

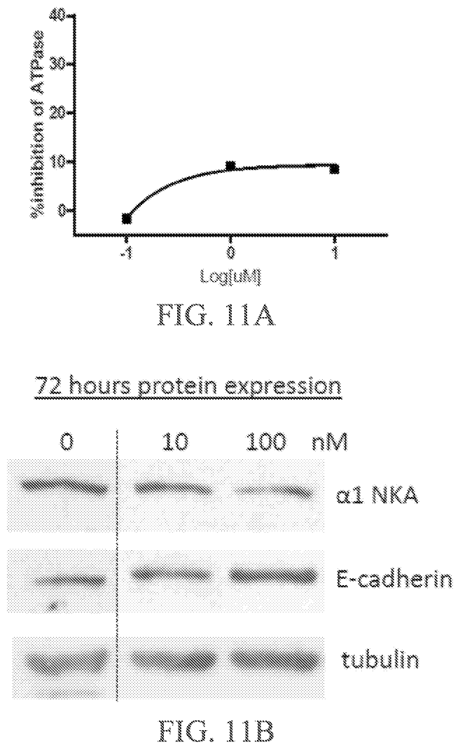

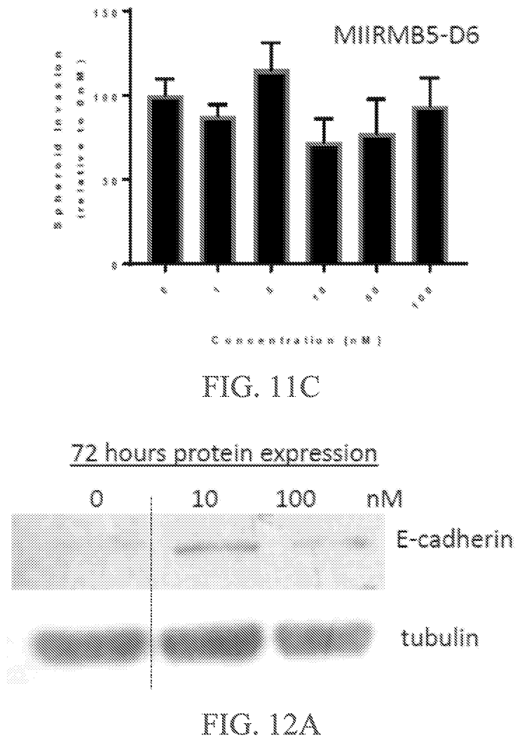

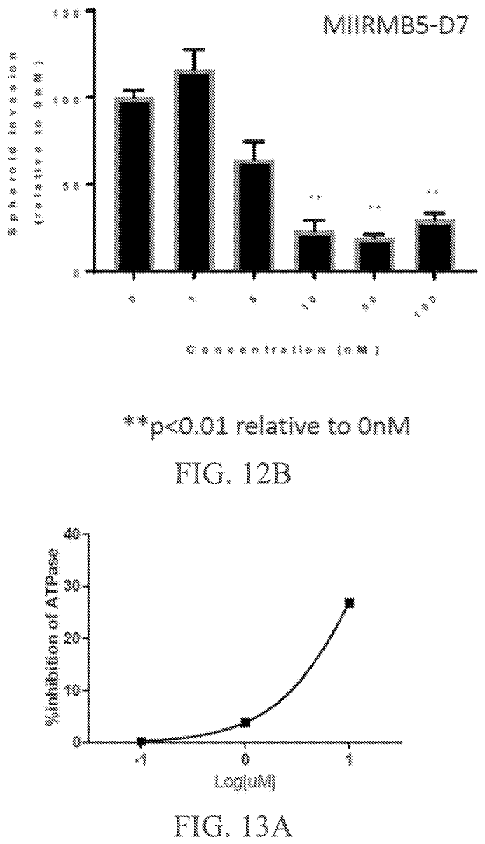

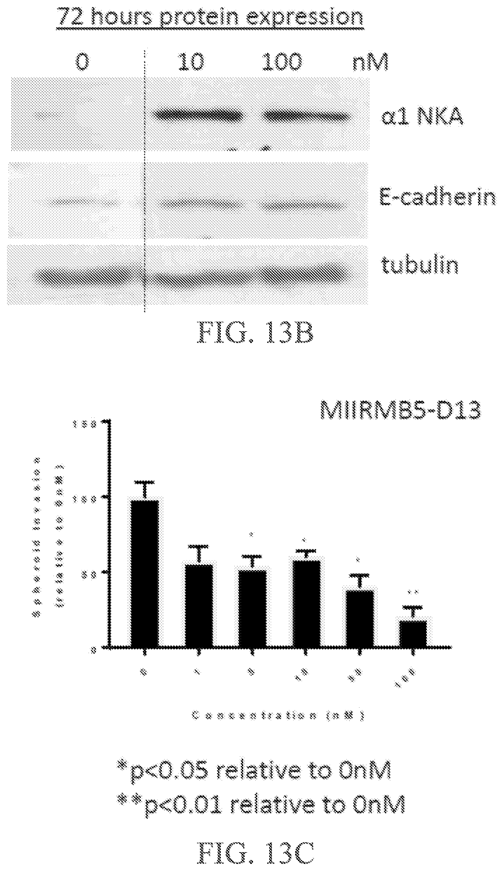

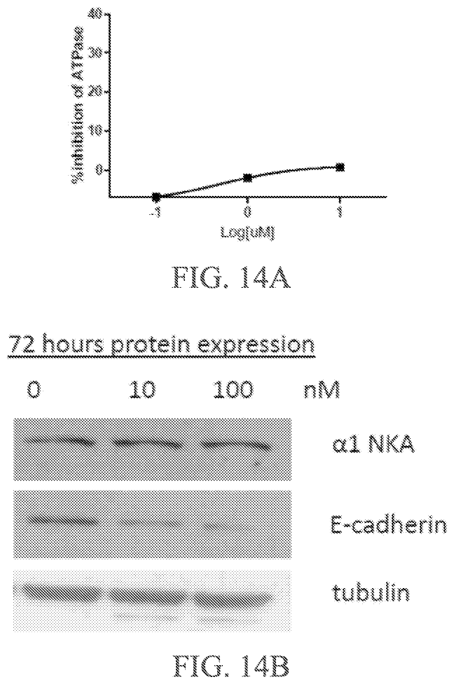

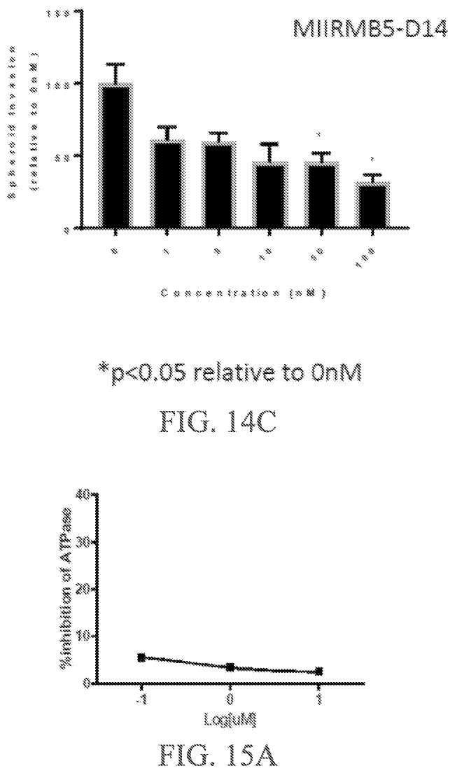

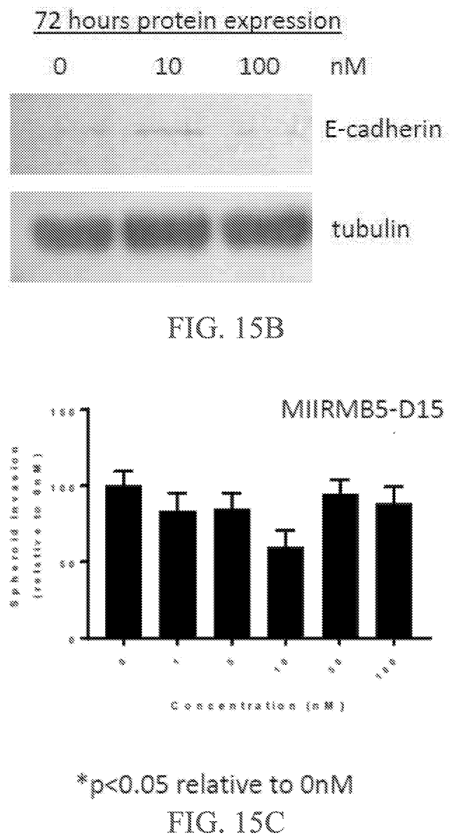

A multi-step assay was further designed to identify which compounds among the MB5 derivatives could be developed as an anti-cancer agent in terms of their potential to stop tumor invasion and growth. This multi-step assay included: (A) an ATPase activity assay- to confirm the binding of derivatives to NKA; (B) an assay to measure inhibition of ouabain-induced ERK activation and NKA endocytosis as well as basal Src/ERK/endocytotic activity in PY-17 cells (NKA/Src inverse agonist); (C) an assay to measure the ability to increase the expression of α1 NKA and E-cadherin level and decrease mesenchymal markers (ZEB1, SNAIL, vimentin) and Myc in cancer cells; (D) a 3D culture to test the anti-invasive and anti-growth potential of spheroids; and (E) an in vivo tumor xenograft model (by transplantation into NOD/SCOD mice). Upon analysis of the results of these experiments, it was believed that some of the MB5 derivative compounds show better or similar properties than MB5 as shown for certain of the compounds in FIGS. 6A-6G. Examples of experiments testing the derivatives are further provided for MIIRMB5 D1 (FIGS. 7A-7B), MIIRMB5 D3 (FIGS. 8A-8C), MIIRMB5 D4 (FIGS. 9A-9C), MIIRMB5 D5 (FIGS. 10A-10C), MIIRMB5 D6 (FIGS. 11A-11C), MIIRMB5 D7 (FIGS. 12A-12B), MIIRMB5 D13 (FIGS. 13A-13C), MIIRMB5 D14 (FIGS. 14A-14C), and MIIRMB5 D15 (FIGS. 15A-15C).

Summary of Examples 1-4

Based on the presented data, the MB5 derivatives were believed to be capable of preventing or reducing tumor metastasis and growth. Further improvements are also possible, based on the SARS data. For example, MIIRMB5 D3 and D4 fall into one class of compounds which are highly effective and more potent than MB5 (about 10-fold) based on their ability to restore NKA and E-cadherin expression and 3D-spheroid invasion data. This property is manifested at very low concentrations of 5-10 nM. On the other hand, MIIRMB5 D13 represents another class of compound which might have similar effect as MB5. The effective dosage of this compound is about 100 nM. Third, all of new compounds have reduced free phenolic groups, which is expected to reduce secondary metabolism, thus improving the ability of the derivatives to be used as effective therapeutic agents. Moreover, based on the results from initial experiments, and without wishing to be bound by any particular theory or mechanism, it was believed that certain structural components may be responsible for MB5 and the derivatives described herein to work as an inverse agonist of α1 NKA/Src receptor antagonists. For instance, it has been observed that MB5, MIIRMB5 D3, MIIRMB5 D4, MIIRMB5 D13, and MIIRMB5 D7 were capable of achieving 80-90% inhibition in the above-described assays, that MIIRMB5 D14 and MIIRMB5 D1 were capable of achieving 50-60% inhibition, and MB7, MIIRMB5 D5, MIIRMB5 D6, and MIIRMB5 D15 were capable of achieving 0-30% inhibition. In this regard, and again without wishing to be bound by any particular theory or mechanism, it was believed that hydroxyl groups at the R4 position of the general formula (I) described herein can be important for activity, while the inclusion of a hydroxyl group or an amine group at the R2 and R3 positions were also useful for achieving inhibition.

Materials and Methods for Examples 5-10

Cell lines. DU145, PC3 and C4-2 cell lines were purchased from and maintained according to ATCC recommendations. Parental DU145 and derived cell lines were cultured in high glucose DMSO medium supplemented with 10% Fetal Bovine Serum and 1% Penicillin/Streptomycin in 37° c. humidified incubator with 5% C02. LLC-PK1, PY17, Y260A, A425P and YFP-α1TCN cells were cultured in the same media and under similar conditions. PC3 and C4-2 cell lines were cultured in RPMI medium with similar conditions as described above. α1 knockdown KD cells were generated from DU145 and C4-2 using a α1 NKA-specific siRNA, as previously described. Knockdown was verified by both qPCR and Western blot analyses. Rat α1 NKA rescued cell lines were generated by transfecting KD cells with a pRC/CMV-α1 AACm1 vector followed by selection with ouabain (2 μM), as previously described. Cells were passaged for three generations without ouabain before conducting experiments.

Mice studies. Animal protocols were approved by an Institutional Animal Care and Use Committee (IACUC) according to NIH guidelines. Tumor xenografts were established by subcutaneous injection of 5×10⁶ cancer cells into the left and right flanks of 10-week-old male NOD/SCID mice (Charles River). Tumor length (L) and width (W) were measured with calipers weekly and tumor volume was estimated as V=(L×W²)/2. After the tumor volumes reached approximately 100 mm³, mice were sacrificed and tumors were harvested. Half of the tumors were used for protein extraction and the other half were digested with collagenase I solution (SIGMA) to isolate cancer cells.

For MB5 treatment, after tumor volume reached approximately 100 mm³, mice were injected peritoneally with DMSO or MB5 at 20 mg/kg or 10 mg/kg daily and further monitored for tumor growth weekly. At the end of treatment period (about 2-3 weeks), tumors were harvested and tumor lysates were analyzed for protein expression by Western blot.

Antibodies and Reagents. Antibodies were sourced and used as follows:

| | | | Catalogue | |
|---|---|---|---|---|
| Antibody | Property | Supplier | number | dilution |
| α1 NKA antibody | Mouse | Developmental Studies | a6f | 1:1000 |
| (α6F) | monoclonal | Hybridoma Bank of | | |
| | | University of Iowa (Iowa) | | |
| PhosphoSrc (Tyr419) | Rabbit | Invitrogen | 44-660G | 1:1000 |
| antibody | polyclonal | | | |
| c-Src B-12 antibody | Mouse | Santacruz Biotechnology | sc-8056 | 1:1000 |
| | monoclonal | | | |
| Rat α1 NKA | Rabbit | Dr. T. A. Pressley (Texas Tech | Not | 1:1000 |
| antibody | polyclonal | University, TX) | applicable | |
| Anti- | Mouse | EMD Millipore | 05-321 | 1:1000 |
| phosphotyrosine | monoclonal | | | |
| antibody, clone 4G10 | | | | |
| c-Myc antibody | | Santacruz Biotechnology | | 1:1000 |
| Anti-tubulin | Mouse | SIGMA | T5168 | 1:2000 |
| antibody | monoclonal | | | |
| Cyclin D1 antibody | Rabbit | Cell Signaling Technology | 2978S | 1:1000 |
| | monoclonal | | | |
| Cyclin E1 antibody | Rabbit | Cell Signaling Technology | 20808S | 1:1000 |
| | monoclonal | | | |
| p53 antibody | Mouse | Calbiochem | OP43 | 1:1000 |
| | pantropic | | | |
| p21 antibody | Rabbit | Santacruz Biotechnology | sc-397 | 1:1000 |
| | polyclonal | | | |
| Phospho MAPK | Rabbit | Cell Signaling Technology | 9101 | 1:1000 |
| antibody | | | | |
| ERK1/2 antibody | Rabbit | Santacruz Biotechnology | sc-94 | 1:1000 |
| | polyclonal | | | |
| Phospho-FAK | Rabbit | Cell Signaling Technology | 3281S | 1:1000 |
| (Tyr576/7) antibody | polyclonal | | | |
| FAK antibody | Rabbit | Cell Signaling Technology | 3285S | 1:1000 |
| | polyclonal | | | |
| Phospho-FAK (Tyr | Rabbit | Cell Signaling Technology | 3283S | 1:1000 |
| 397) antibody | polyclonal | | | |
| Anti-Na+/K+- | Mouse | EMD Millipore | 05-382 | 1:1000 |
| ATPase β1 antibody | monoclonal | | | |
| clone 464.8 | | | | |
| E-cadherin (24E10) | Rabbit | Cell Signaling Technology | 3195S | 1:1000 |
| antibody | monoclonal | | | |
| Anti β-catenin | Mouse | BD Bioscience | 610153 | 1:1000 |
| antibody | monoclonal | | | |
| ZO-1 antibody | Rabbit | Thermo-Fisher Scientific | 61-7300 | 1:1000 |
| | polyclonal | | | |
| ZO-2 antibody | Rabbit | Thermo-Fisher Scientific | 38-9100 | 1:1000 |
| | polyclonal | | | |
| Occludin antibody | Mouse | Thermo-Fisher Scientific | 33-1500 | 1:1000 |
| (OC-3F10) | monoclonal | | | |
| SNAIL (C15D3) | Rabbit | Cell Signaling Technology | 3879S | 1:1000 |
| antibody | monoclonal | | | |
| TCF8/ZEB1 | Rabbit | Cell Signaling Technology | 3396S | 1:1000 |
| antibody | monoclonal | | | |
| Vimentin (D21H3) | Rabbit | Cell Signaling Technology | 5741S | 1:1000 |
| antibody | monoclonal | | | |
| MMP-2 antibody | Rabbit | Cell Signaling Technology | 87809S | 1:1000 |
| | monoclonal | | | |
| MMP-9 antibody | Rabbit | Cell Signaling Technology | 13667S | 1:1000 |
| | monoclonal | | | |
| PCNA antibody | Mouse | Santacruz Biotechnology | sc-56 | 1:2000 |
| | monoclonal | | | |
| Lamin B antibody | Goat | Santacruz Biotechnology | sc-6216 | 1:1000 |
| | polyclonal | | | |
| β actin antibody | Mouse | Santacruz Biotechnology | sc-47778 | 1:1000 |
| | monoclonal | | | |
| SLUG (C19G7) | Rabbit | Cell Signaling Technology | 9585S | 1:1000 |
| antibody | monoclonal | | | |
| N cadherin (D4R1H) | Rabbit | Cell Signaling Technology | 13116S | 1:1000 |
| antibody | monoclonal | | | |

All reagents were obtained from SIGMA except FAK inhibitor (Millipore Catalogue No. 324877).

Western blot, Immunoprecipitation and Immunostaining. Cells were grown to 100% confluency and Western blot were performed as described before. Images were quantified with ImageJ software from NIH. Immunoprecipitation studies were performed as described before. α1 NKA immunostaining was performed by growing cells on sterilized coverslips in 6 well tissue culture plates and permeabilization/fixation with ice-cold methanol followed by blocking with 5% horse serum and 0.1% Triton X-100 in 1× Phosphate buffered saline (PBS) for 30 minutes. Coverslips were then stained with an anti-α1 NKA antibody (Millipore, Cat #05-369) at 1:100 dilution in 1% BSA (Bovine Serum Albumin) containing 1×PBS solution for overnight. Next day after three washes, coverslips were stained with Alexa Fluor 488 conjugated anti-mouse secondary antibody (Thermo-Fisher) for 1 hour, washed extensively and then imaged using a fluorescent microscope with GFP filter or confocal microscope (LEICA-DMIRE2). E-cadherin and occludin immunostaining was performed in a similar manner. Phospho-Src, phospho ERK1/2 immunostaining were performed as described before and images were taken with a LEICA DMIRE2 confocal microscope.

RNA extraction, cDNA synthesis and qPCR. Total RNA from cells were extracted using RNeasy minikit from Qiagen. Same amount of RNA was used to synthesize cDNA with Superscript III First-Stand Synthesis SuperMix for qRT-PCR (ThermoFisher). qPCR was performed as described before.

Boyden chamber assay. Different sub-clones were grown up to 100% confluency and then gently trypsinized with 0.05% trypsin-EDTA and 100,000 cells were plated in upper chamber of a 0.8μ pore containing transwell filter with 0.5% FBS containing media. Full serum (10% FBS) containing media was added to the lower chamber as chemoattractant. After 16 hours, a colorimetric assay was used to determine the migration of cells to the lower side of the filter, with absorbance read at 590 nm. Briefly, lower sides of the transwell were washed with 1×PBS and then fixed with ice cold methanol for 10 minutes. After gentle washes with 1×PBS, lower side of the filter were stained with Crystal violet solution (0.5% Crystal Violet in 20% Ethanol), washed and the stain was extracted with methanol. Result was normalized against migration of sub-clone 5 cells. For cell migration in presence of pharmacological compounds, cells at 100% confluency were pretreated for 24 hours with or without MB5, PP2 (Sigma-Aldrich, Catalogue No P0042) or FAK inhibitor I (Millipore, Catalogue No. 324877) and then assay was performed as described above.

3D culture—spheroid formation assay. 10,000 cells/well were plated on top of a solidified 3D matrix composed of 1:1 Collagen (Thermofisher Cat #A1048301) and Matrigel (Corning Cat #354234) in 6 well plates and allowed to form spheroids for a week. Full serum (10% FBS) containing DMEM media was added on top of the matrix and was changed every two days. After 7 days, spheroid formation was recorded using a phase contrast microscope fitted with a camera.

3D culture-spheroid invasion assay. Cells were trypsinized gently (0.05% Trypsin-EDTA) and diluted at 1000 cells in 20 μl of full serum media for DU145 and C4-2 derived sub-clones or 500 cells in 20 ul for PC3 cells and allowed to form spheroids using hanging drop technique for 3 days, as described previously. Generated spheroids were then embedded into 3D matrix (not yet solidified at the time of experiment), as described in the previous section, on top of a 1% solidified agarose coating in 48 well plates. Spheroids were monitored from day 1 to different time points and images were taken using a phase contrast microscope fitted with camera, at same settings. Spheroid growth or invasion area was quantified using ImageJ software from NIH. The following formulas were used for quantitation of spheroids:

Spheroid invasion=(Invasion area/Spheroid area)*100

Spheroid growth=(Spheroid area at Day 3 or 7-Spheroid area at 16 hours)/Spheroid area at 16 hours

For drug treatment, compound was diluted into media, from stock solution. For MMP secretion, conditioned media for 3 days were collected from top of matrix and assessed for MMP secretion by Western blot using Matrix Remodeling Antibody Sampler kit from Cell Signaling Technology (Cat #73959).

α1 NKA endocytosis assay. Endocytosis assay was performed as described before.

Spheroid aggregation in ultralow attachment plates. 5000 cells were plated in a 6 well ultralow attachment plate from Corning and allowed to form spheroids spontaneously for a week. Cell aggregates were then immunostained for E-cadherin expression using a monoclonal anti-E-cadherin antibody (Santacruz Biotechnology, Catalogue No.sc-71008), followed by Alexa-Fluor 488 anti-mouse secondary antibody (Thermo-Fisher).

CellProliferation assay. Cell proliferation assay was performed by plating 5000 cells per well of 96 well plate and cell proliferation was analyzed by Cell Titer Glo Assay (Promega) according to the manufacturer's instructions.

ATPase activity assay. ATPase activity assay was performed as previously described.

Cell death assays. MTT assay was performed as described before. Cell Titer glow assay was performed in 96 well plates according to manufacturer's recommendation (Promega, Cat #G7570).

Statistical analysis. Data are shown as mean+/−SEM. Student's t-test was used to compare two individual groups and one-way analysis of variance (ANOVA) followed by multiple comparison analysis via Dunnett's or Sidak's test was used when comparing more than two groups. Graphs were prepared and analyzed using GraphPAD PRISM software. Statistical significance was accepted at p value less than 0.05.

### Example 5—Knock-Down of α1 NKA Induces EMT and Promotes PCa Cell Migration and Invasion

Alteration of NKA cellular distribution in PCa cells is secondary to an increase of al NKA receptor endocytosis. This mechanism is NKA-specific and can be modified pharmacologically by modulating its receptor function. Cardiotonic steroids (CTS) are the archetypal and best-studied NKA ligands. They bind to and inhibit the enzymatic activity of NKA by stabilizing the protein in its E2P conformation. Because E2P represents an active conformation for Src and α1 NKA interaction, CTS such as ouabain are agonists of the receptor α1 NKA/Src complex. Accordingly, these compounds stimulate protein and lipid kinases, increase Reactive Oxygen Species (ROS) production and induce the endocytosis of α1 NKA. A high throughput screening platform was developed to identify novel non-CTS α1 NKA ligands and assess their molecular actions on the signaling function of α1 NKA as either agonists or inhibitors. Using this platform, a group of small molecules with a xanthone backbone were identified that bind NKA but do not provoke NKA-mediated signal transduction. Hence, it was surmised that this family of compounds could be modulators of NKA cell surface expression and inhibitors of EMT in metastatic PCa cells.

Using a combination of NKA gene targeting, EMT markers analyses, pharmacological characterization, and functional assays in 3D spheroids and tumor xenograft models, the studies presented herein suggest that MB5, a small molecule inverse agonist of NKA receptor function, can block metastasis and reduce tumor growth by reversing EMT in PCa.

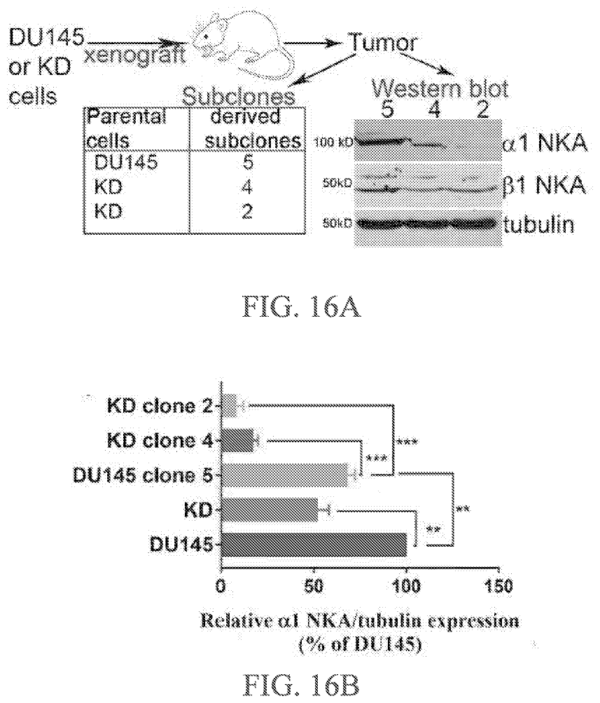

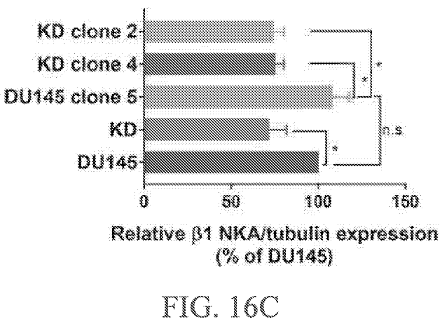

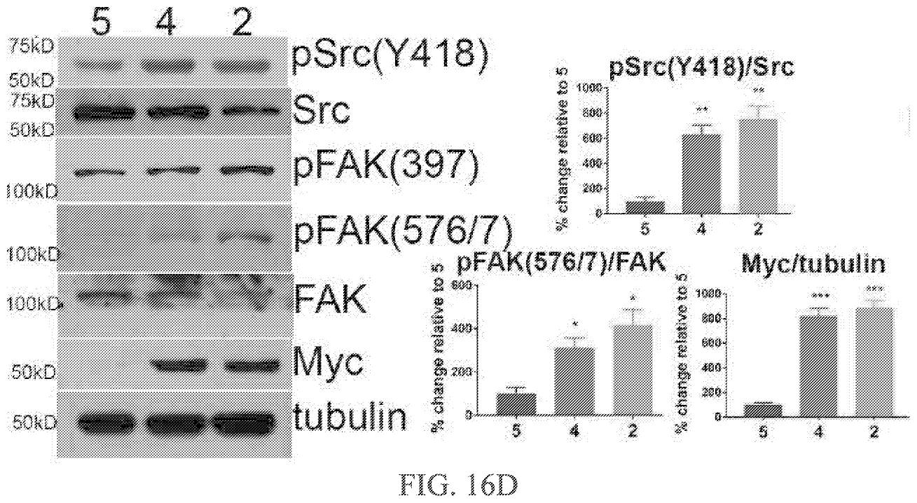

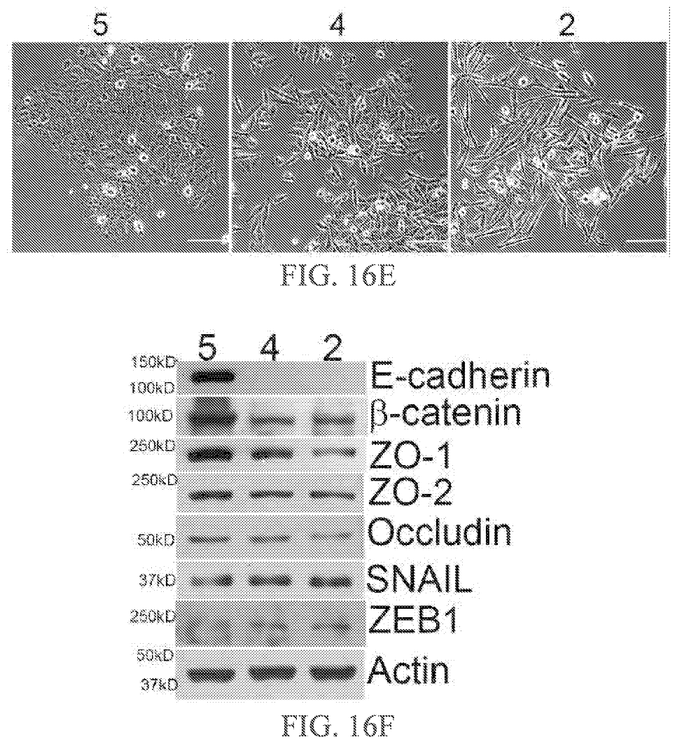

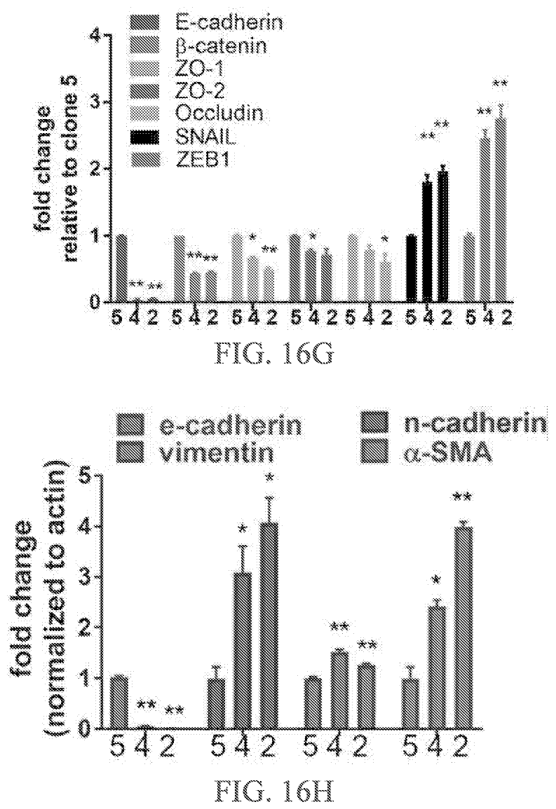

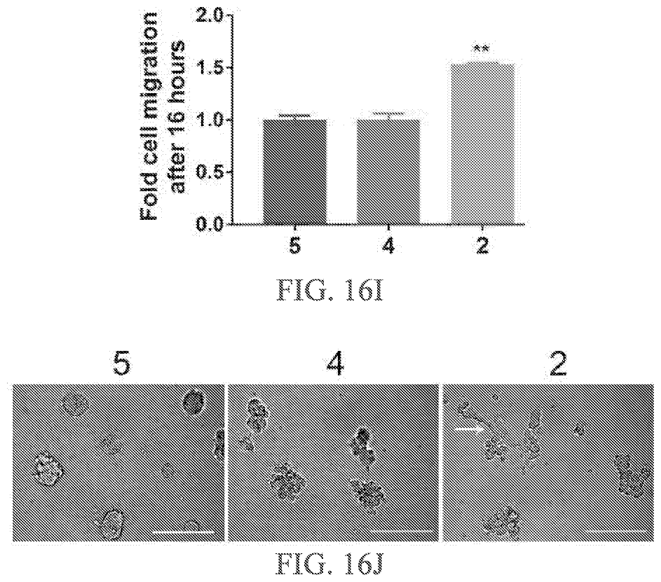

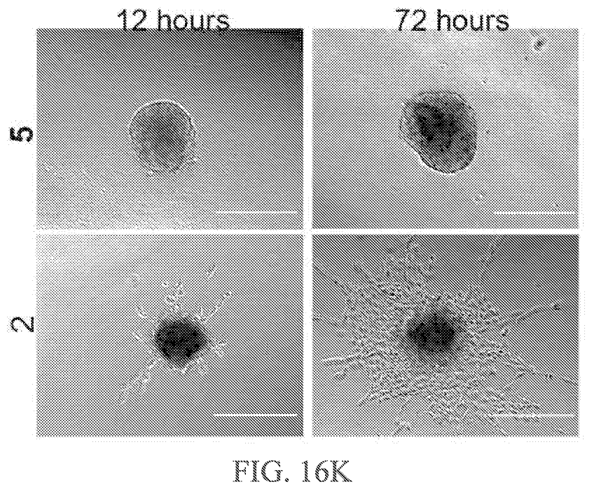

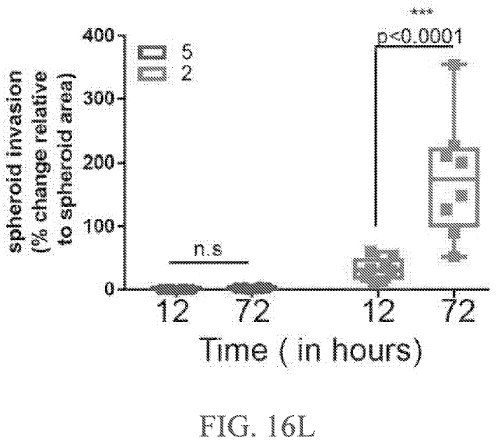

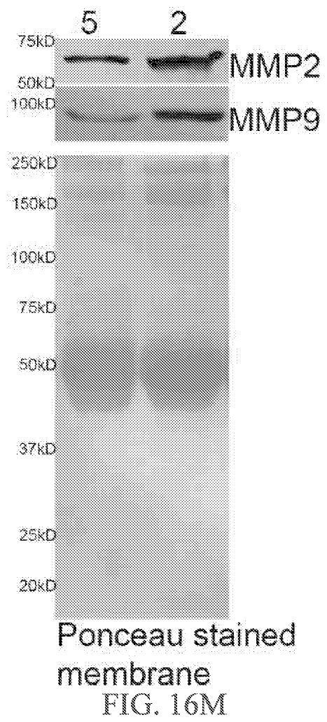

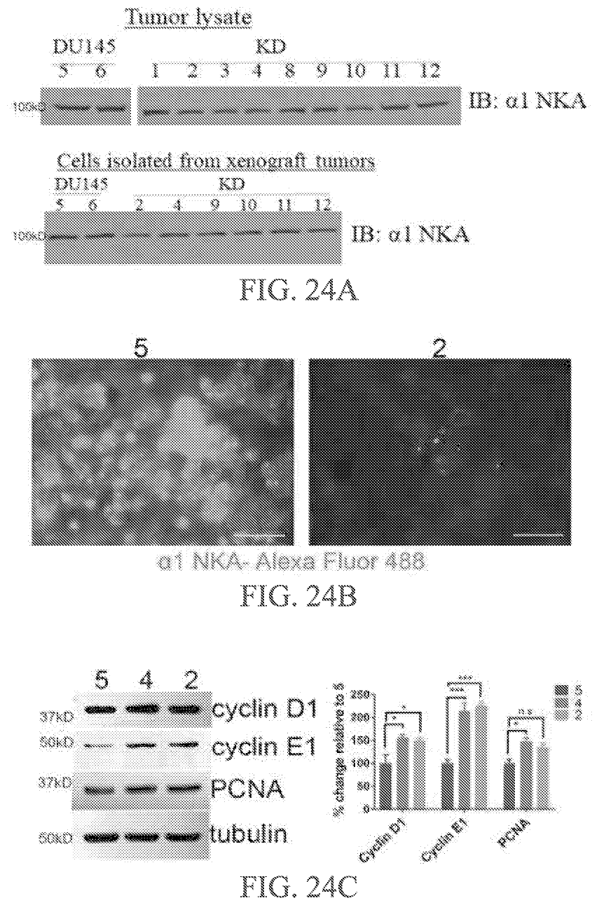

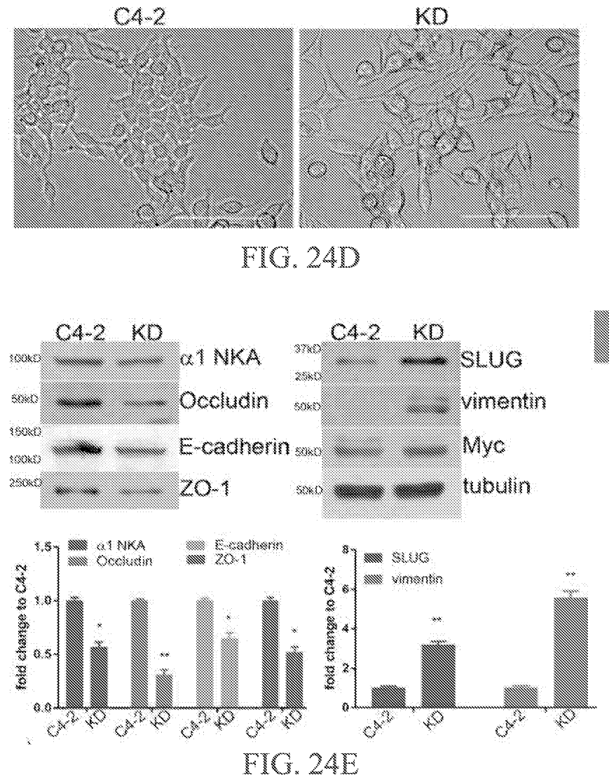

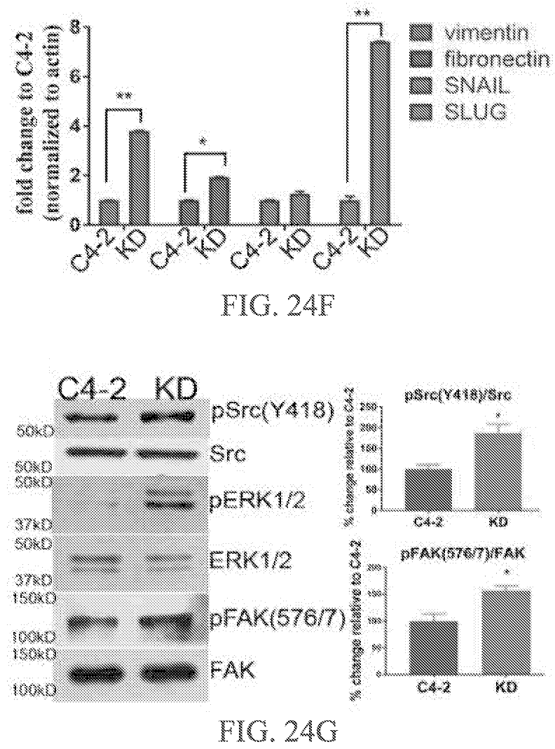

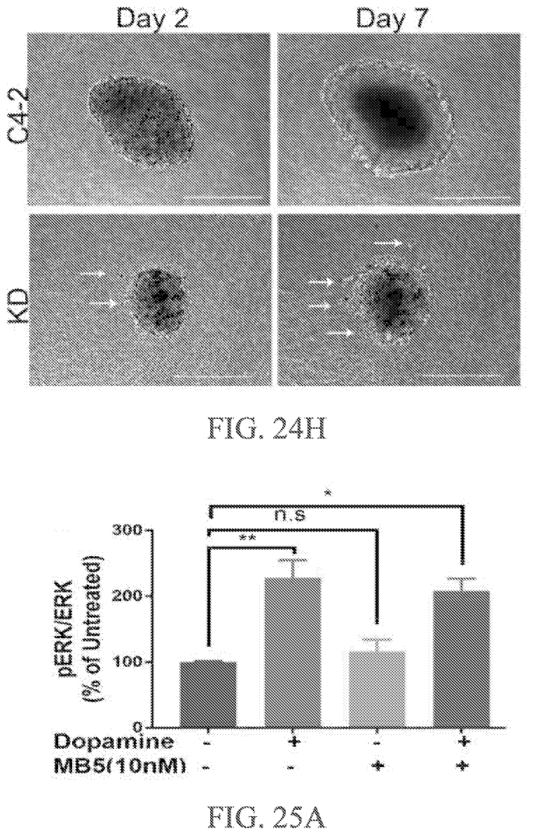

First, to experimentally assess the impact of loss of NKA expression on tumor growth and metastatic potential, DU145 and DU145-derived NKA knockdown (KD) PCa cells (˜50% reduced by RNAi) were xenografted into NOD/SCID mice to generate tumors (FIG. 16A). All tumors grew locally at the injection sites except one tumor from KD cells, which metastasized to the bones. Cell lysates were prepared from one half of each tumor sample, whereas tumor cells were isolated from the other half by enzymatic digestion. FIG. 24A shows total α1 NKA protein expression in each tumor sample. Subclone 5 (isolated from a DU145 xenograft) as well as subclones 4 and 2 (isolated from KD xenografts) were selected for further comparison. Western blot analyses (FIG. 16B) showed that α1 NKA expression was significantly reduced in the subclones compared to the parental cell lines. Subclone 2, a cell line isolated from the only bone metastatic tumor, exhibited the lowest expression of α1 NKA (˜80% reduced expression compared to KD cells). Expression of 31 NKA, which also has a tumor suppressor function, was only modestly reduced in subclones 4 and 2 compared to subclone 5 (FIG. 16C). Reduction in α1 NKA expression was further verified by immunostaining subclones 5 and 2 with a monoclonal anti-α1 NKA antibody (FIG. 24B). Consistent with previous findings, decrease in α1 NKA expression was inversely associated with activation of Src kinase and its effector protein FAK (phosphoprotein/total protein ratios) and Myc expression (FIG. 16D). This was accompanied by a modest increase in expression of cell cycle proteins cyclin D1, E1 and PCNA (cell proliferation marker) (FIG. 24C).

In 2D cell culture, severely decreased expression of α1 NKA was associated with a change in phenotype from epithelial to mesenchymal (fibroblastic) morphology (FIG. 16E). This effect was most pronounced in subclone 2, which expressed the least amount of α1 NKA and exhibited an extreme spindle shape and loss of cell-cell attachment. Subclone 5, with the highest NKA expression, retained the typical cobblestone epithelial phenotype of DU145 cells, whereas subclone 4 displayed an intermediate phenotype. Further Western blot analyses of EMT signature markers (FIGS. 16F-16G) revealed that expression of several epithelial markers like E-cadherin, β-catenin, ZO1/2 and occludin were significantly downregulated in subclones 4 and 2. This was accompanied by a significant increase in mesenchymal proteins SNAIL and ZEB1, thus consistent with an EMT phenotype. This EMT phenotype was further verified by qPCR of EMT markers such as E-cadherin, vimentin, or N-cadherin (FIG. 16H). Because a gain of EMT phenotype is associated with increased migratory capability, a Boyden chamber assay was conducted (FIG. 16I), which revealed that subclone 2 cells migrated significantly faster than both subclones 5 and 4.

EMT is associated with loosened cell-cell contact allowing metastatic dispersion of cancer cells, therefore experiments were undertaken to test if loss of α1 NKA contributes to decreased cell-cell adhesion and increased invasion. In 3D culture (FIG. 16J), subclone 5 formed compact homogenous spheroids after 7 days, indicating its capability to form strong intracellular adhesion. In contrast, subclones 4 and 2 formed loose grape-like stellate spheroids and also invaded into the matrix during this time, consistent with their inability to maintain intracellular adhesion under reduced α1 NKA expression. The invasive capability of the subclones was next tested using a spheroid invasion assay. As illustrated in FIG. 16K, compact spheroids were generated by the hanging drop technique and embedded in a 3D matrix. Cells from subclone 2 spheroids invaded into the matrix as early as 12 hours and formed extensive invasive structures by 72 hours. In contrast, subclone 5 spheroids did not invade into the matrix even after 72 hours of culture (FIGS. 16K-16L). To complement these findings, matrix metalloproteinase (MMP) secretion into 3D culture media was measured as an indicator of PCa cells' ability to degrade extracellular matrix and enable metastasis. Subclone 2 secreted significantly more MMP2 and MMP9 into the culture media than subclone 5, as measured by Western blot analyses of conditioned media (FIG. 16M).

The above cell KD models were derived from the DU145 cell model of CRPC. It was therefore important to verify the proposed concept in another PCa cell model. Accordingly, al NKA expression was knocked down in the C4-2 cell CRPC model using RNAi. Although the knockdown efficiency was only 40% (FIG. 24E), it resulted in an EMT phenotype as evident from the morphological change from the tightly clustered epithelial colony formed by parental C4-2 cells to the significant loss of cell-cell attachment and fibroblastic morphology of α1 NKA KD cells (FIG. 24D). This was accompanied by significant reduction in epithelial markers such as occludin, E-cadherin and ZO-1 expression and upregulation of mesenchymal markers in the KD cells (FIGS. 24E-24F). In agreement with a Src kinase regulatory role for α1 NKA, Src kinase and its effector proteins FAK and ERK were significantly activated in the KD cells (FIG. 24G). The spheroid invasion assay revealed that reduced expression of α1 NKA enabled KD cells to invade into the matrix in 2 days, which is in sharp contrast to parental C4-2 spheroids that exhibited no invasion even after 7 days in culture (FIG. 24H).

### Example 6—α1 NKA Rate of Endocytosis Correlates with EMT in PCa Cell Lines

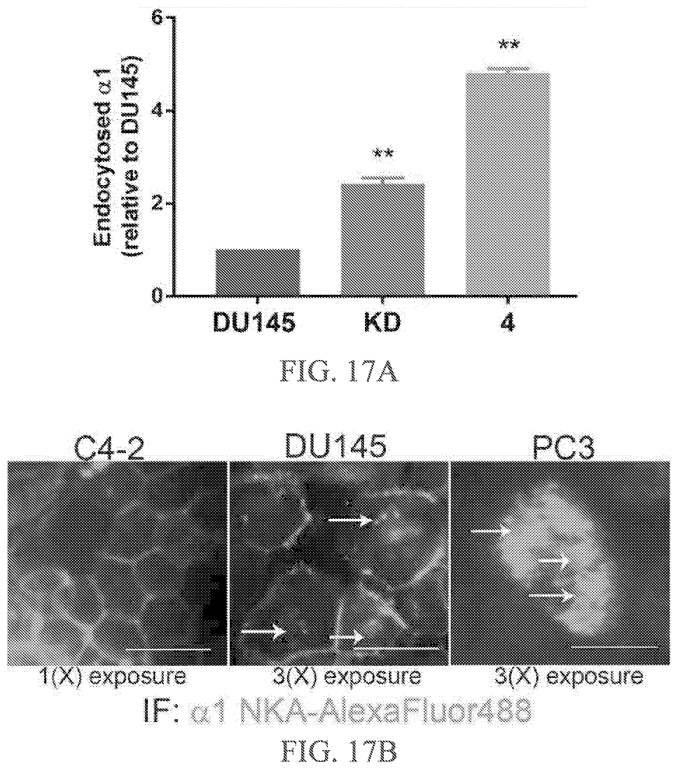

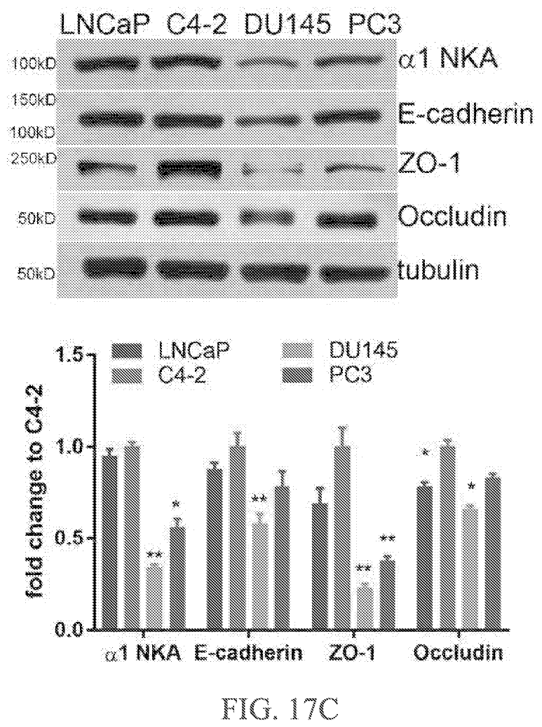

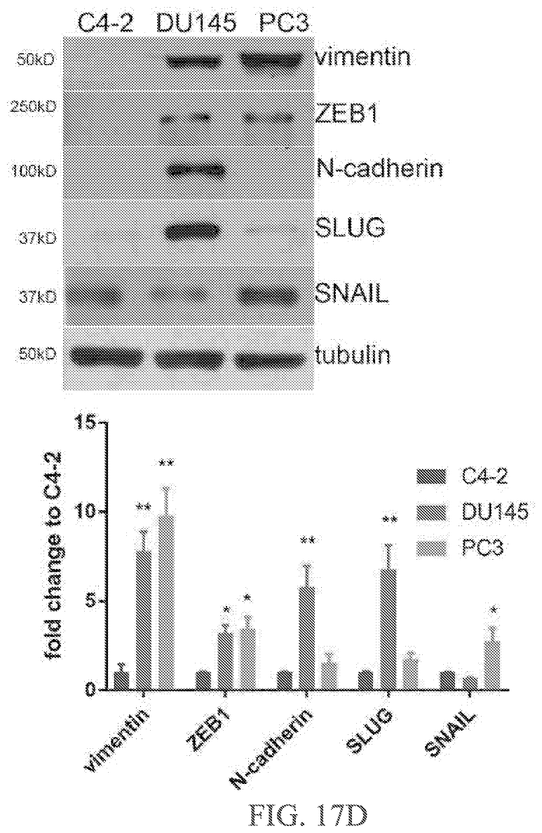

Previous studies have shown that NKA expression in PCa cells is regulated by a posttranslational mechanism (increased endocytosis) rather than a transcriptional mechanism, which is supported by findings from other laboratories. In the present study, biotinylation assays showed that the rate of endocytosis of α1 NKA was indeed highest in the aggressive subclone 4 compared to the parental DU145 and the KD cells from which it was derived, by about 5 fold and 2 fold, respectively (FIG. 17A). This increase in endocytosis was inversely proportional to their total α1 NKA expression (FIG. 16B). To determine whether changes in α1 NKA expression correlate with an EMT phenotype, α1 NKA expression was analyzed in four PCa cell lines by Western blot and immunofluorescence. As shown in FIG. 17C, DU145 and PC3 cell lines derived from distant metastatic sites express a significantly lesser amount of α1 NKA than LNCaP, a lymph node metastatic cell line, or its derivative C4-2. Moreover, immunofluorescence analyses (FIG. 17B) revealed that whereas all α1 NKA signal was localized to the plasma membrane in C4-2 cells, a significant amount of α1 NKA resided in intracellular compartments in DU145 and almost all of the signal was observed in the cytosol for PC3 cells. This was associated with an upregulated EMT phenotype in DU145 and PC3 as compared with C4-2 (FIGS. 17C-17D), thus further supporting the contention that α1 NKA rate of endocytosis correlates with EMT in PCa cells.

### Example 7—Overexpression of α1 NKA is an Approach to Counter Tumor Growth in the NOD/SCID Mouse Model

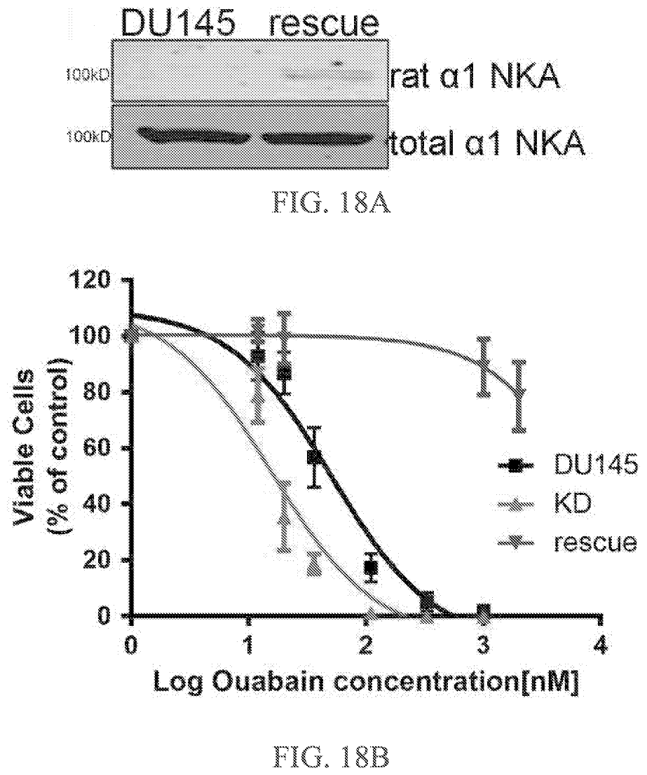

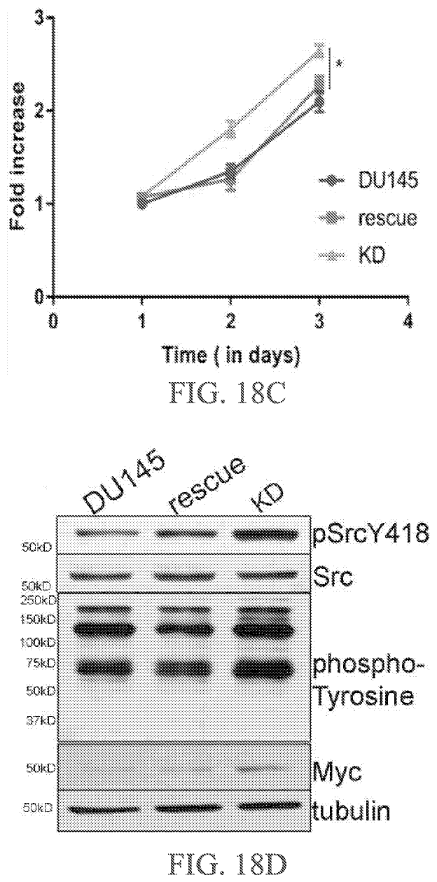

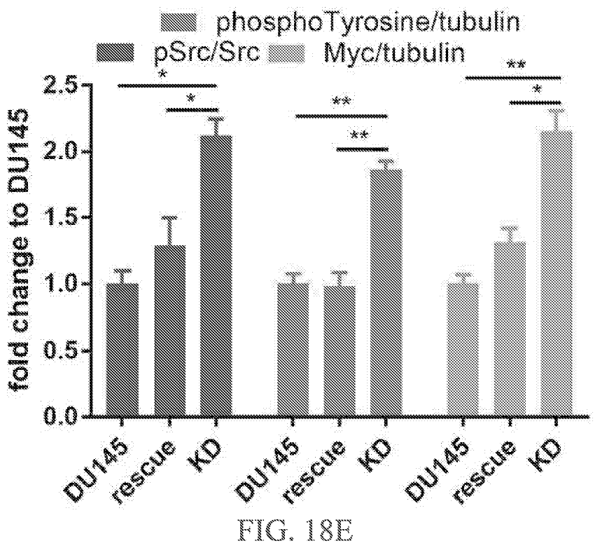

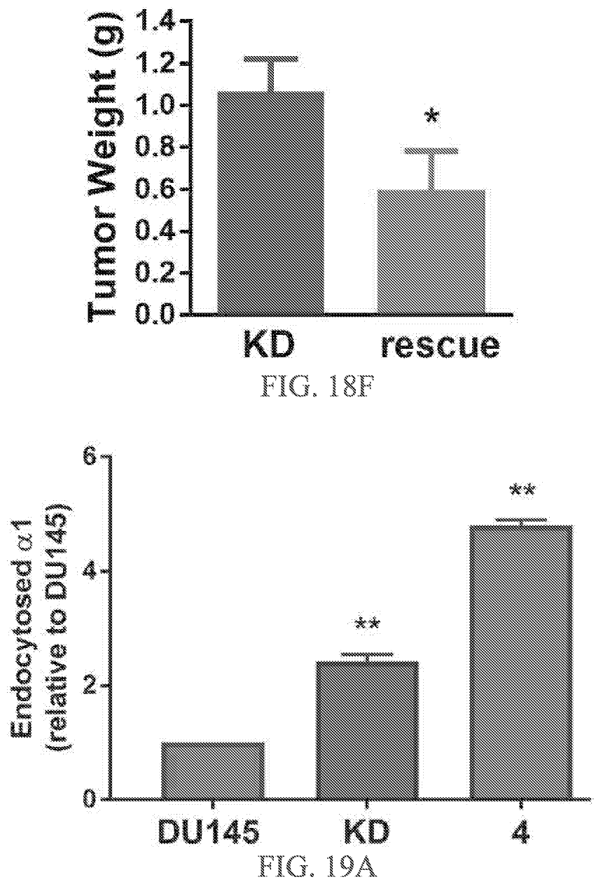

Since the loss of α1 NKA expression contributes to tumor progression, it was next tested whether genetic rescue of its expression counters tumor growth in a xenograft mouse model. Specifically, the KD cells derived from DU145 were rescued with a murine α1 NKA construct-containing expression vector. Rat α1 NKA expression was verified by Western blot analyses (FIG. 18A). In addition, taking advantage of the well-known low affinity of rodent NKA α1 to ouabain compared to human, successful rescue was functionally confirmed by acquired resistance to ouabain-induced cell death using a MTT assay (FIG. 18B). As shown in FIG. 18C, α1 KD cells had increased cell proliferation rate in comparison with parental DU145 cells, but rat α1 rescue decreased the cell proliferation rate to a level similar to DU145. This anti-proliferative effect was correlated with decreased Src activation, Myc expression and total protein tyrosine phosphorylation in α1 NKA rescued cell line in comparison with the KD cells (FIGS. 18D-18E). Finally, when xenografted into NOD/SCID mouse model, rat α1 rescued cells formed significantly smaller tumors than those from KD cells (FIG. 18F). This dataset indicated that rescue of α1 NKA expression could represent a novel mechanism for preventing PCa progression. Based on this, a cell based assay was implemented to identify pharmacological agents that can inhibit α1 NKA endocytosis.

### Example 8—Identification of MB5, an Inverse Agonist that Targets α1 NKA/Src Signaling

As archetypal agonists of NKA receptor function, ouabain and other CTS stimulate cellular signaling by activating α1 NKA/Src signalosome complex, which results in its endocytosis. It was reasoned that compounds that inhibit CTS induced signaling could function as inverse agonists of this signalosome complex and prevent α1 NKA endocytosis. A group of hydroxyxanthones were thus tested that were identified as a new class of Na/K-ATPase ligands using a high throughput in vitro screening platform and a library of 2600 structurally diverse chemicals. Structure Activity Relationship (SAR) studies of these new ligands revealed a range of pharmacological potency on NKA enzymatic activity, with at least one compound without detectable CTS-like activating (agonist) effect on NKA receptor signaling. Further pharmacological characterization of this small molecule, MB5 (FIG. 1), revealed a biphasic NKA inhibition curve with a high affinity component (nM), which represented only 20% of Na/K-ATPase activity, and a low affinity component with an IC50 of 10 μM (FIG. 1B).

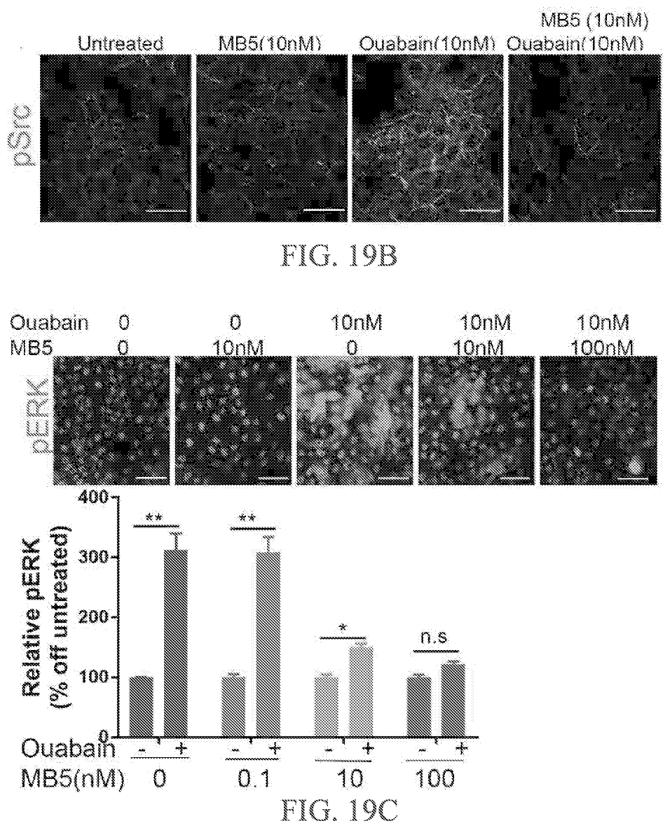

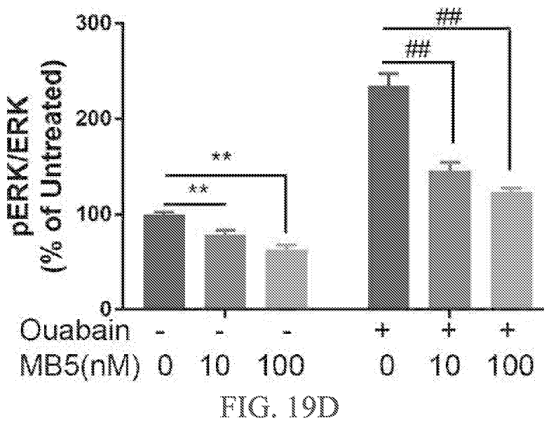

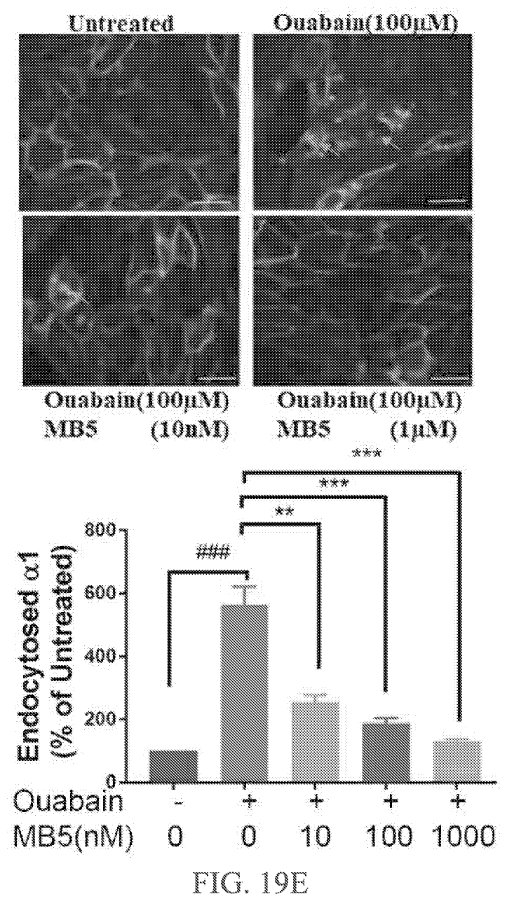

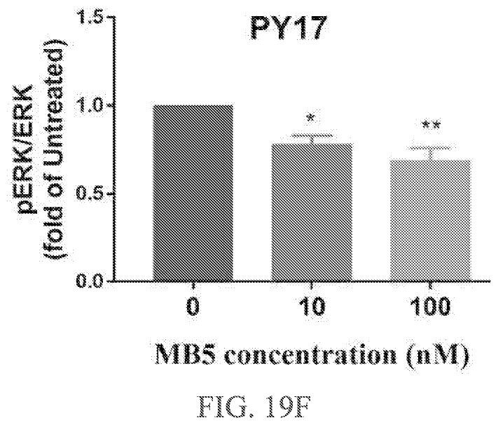

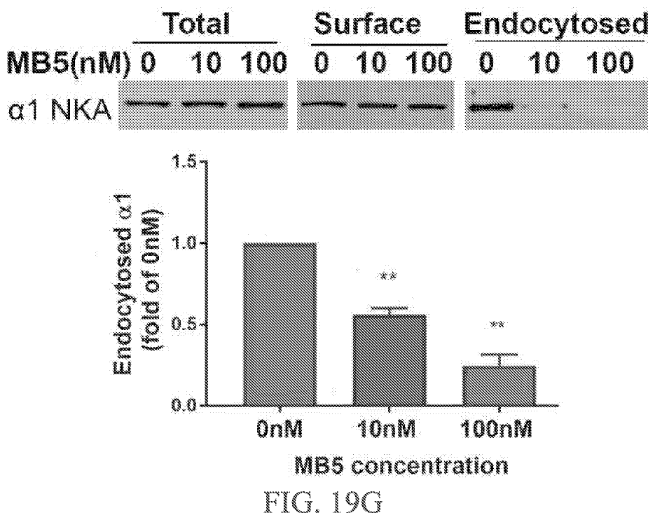

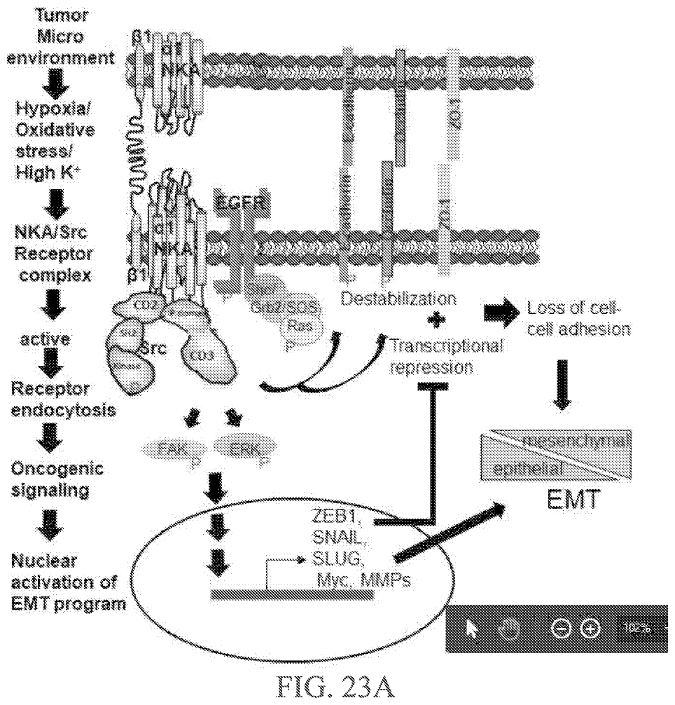

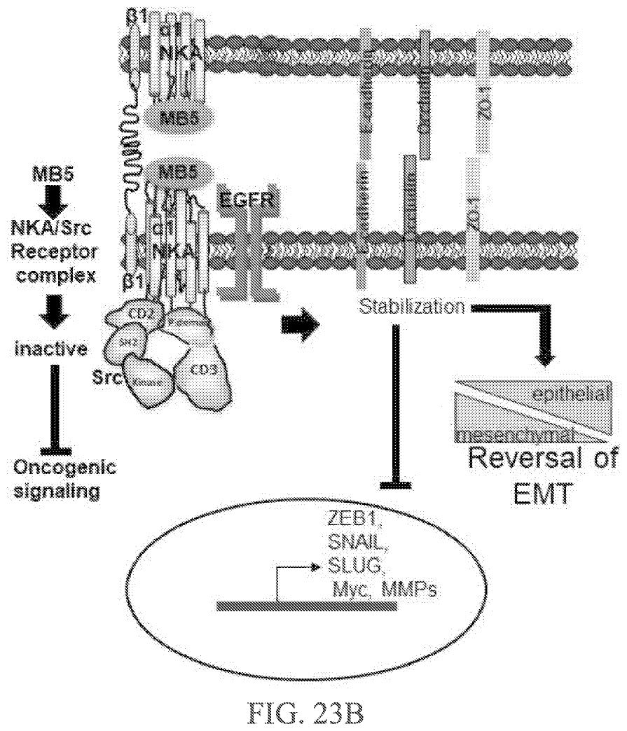

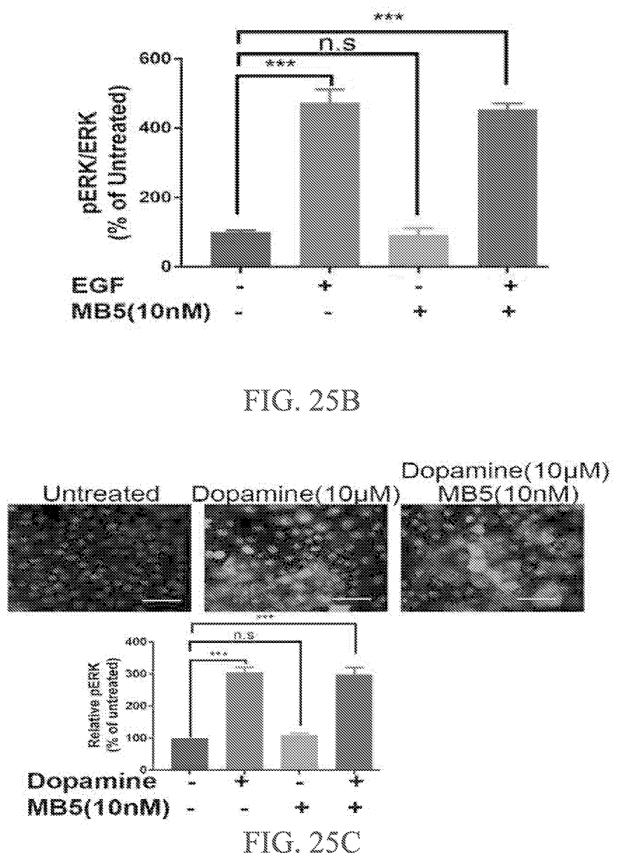

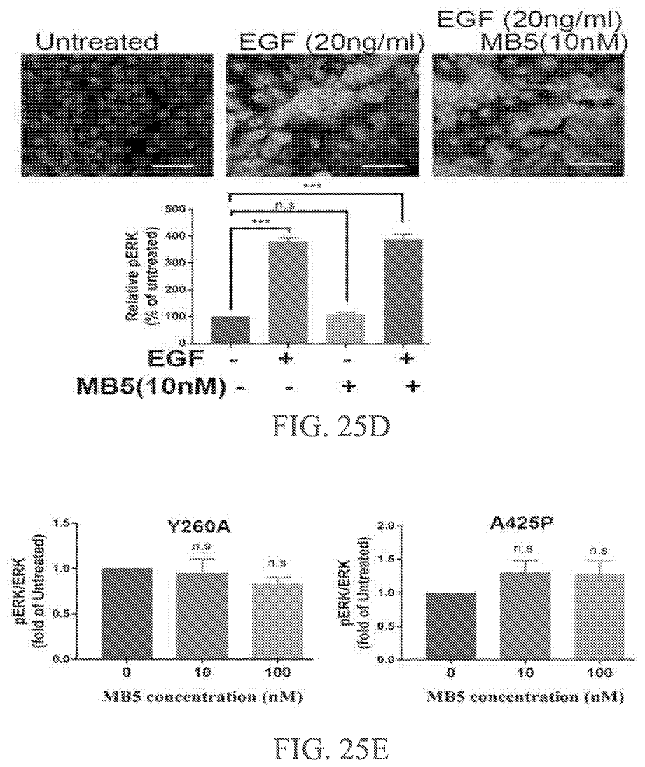

Characterization of MB5 activity on the NKA receptor function known as the NKA/Src binary receptor mechanism was conducted in a pig kidney epithelial cells (LLC-PK1)-based platform using immunostaining and Western blot analyses. The binary NKA/Src receptor model summarized in FIGS. 23A-23B has been shown to regulate a series of downstream signaling events that include ERK phosphorylation and endocytosis in LLC-PK1 cells and a number of other cells. According to this model, under normal basal conditions, most NKA/Src binary receptors adopt a conformation whereby Src binds to NKA through two defined sites of interaction and is kept inactive. The change of NKA conformation that occurs upon binding of agonists such as ouabain, results in the release and activation of the Src kinase domain from the “Naktide” site of NKA CD3, while the other interaction (constitutive) persists between NKA CD2 and the SH2 domain of Src. As presented in FIGS. 19B-19D, MB5 (10-100 nM) did not activate NKA receptor function under baseline condition, but dose-dependently inhibited Src and ERK activation induced by the prototypic NKA agonist ouabain. Accordingly, MB5 was also a potent inverse agonist of ouabain-induced α1 NKA endocytosis as confirmed via confocal microscopy using a cell line expressing YFP tagged-murine α1 NKA (FIG. 19E). This effect was specific to NKA-mediated signaling, as MB5 failed to inhibit dopamine- and EGF-induced ERK activation (FIGS. 25A-25D). In LLC-PK1-derived cells with low NKA receptor constitutive activity due to substantial reduction (over 80%) of α1 NKA expression by siRNA (PY17 cells), increased basal Src and ERK activity is observed due to the abnormally high amount of Src kinase that remains in an active conformation. Increased basal Src and ERK activity is also observed in LLC-PK1-derived cells with reduced NKA receptor constitutive activity that result from mutations in either domain of interaction with Src without alteration of the ion pumping activity (e.g. A425P on the Naktide sequence, or Y260A on the CD2 domain). As shown in FIG. 19F, MB5 inhibited ERK phosphorylation in a dose-dependent manner in PY17. Based on the inverse agonism observed in the presence of ouabain, MB5 effect in PY17 is best explained by a stabilization of the remaining NKA receptors in an inactive conformation, which in turn normalized basal phospho ERK in those cells. In contrast, MB5 did not reduce high levels of ERK phosphorylation in A425P and Y260A (FIG. 25E), suggesting that MB5 specifically targets the NKA/Src signaling branch of NKA receptor function. Critically, as in PY17, MB5 inverse antagonism also applied in PCa cells with increased Src kinase activity due to low NKA levels, where MB5 treatment abolished α1 NKA endocytosis in subclone 4 cells in a dose-dependent manner (FIG. 19G).

### Example 9—MB5 Reverses EMT in PCa Cells and Reduces their Metastatic Potential

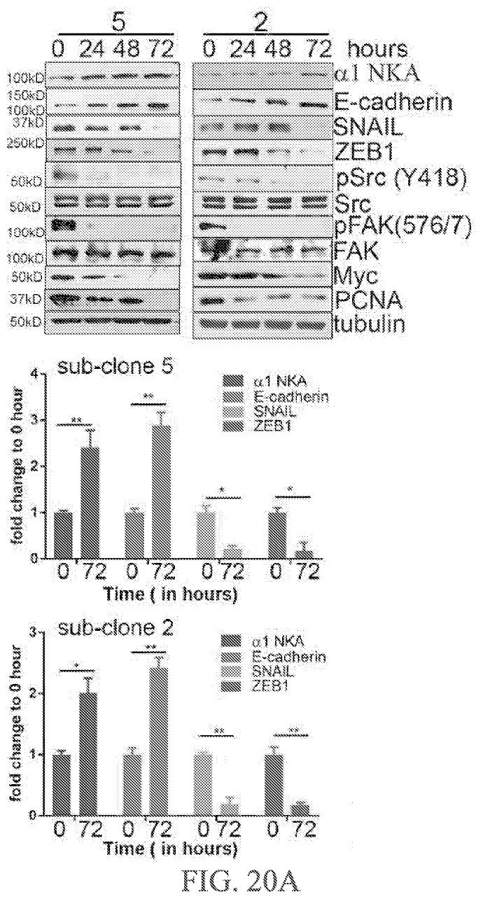

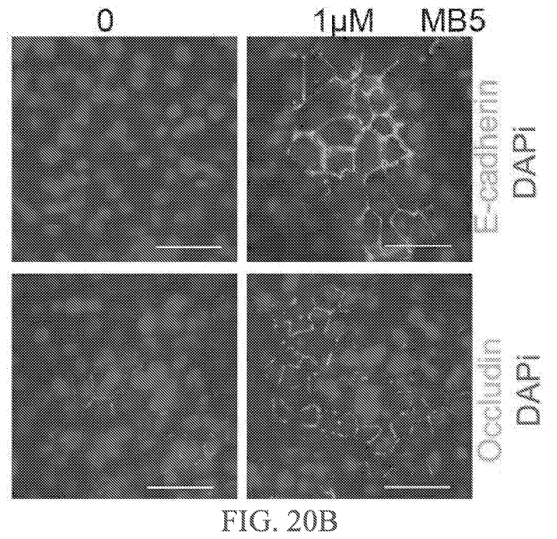

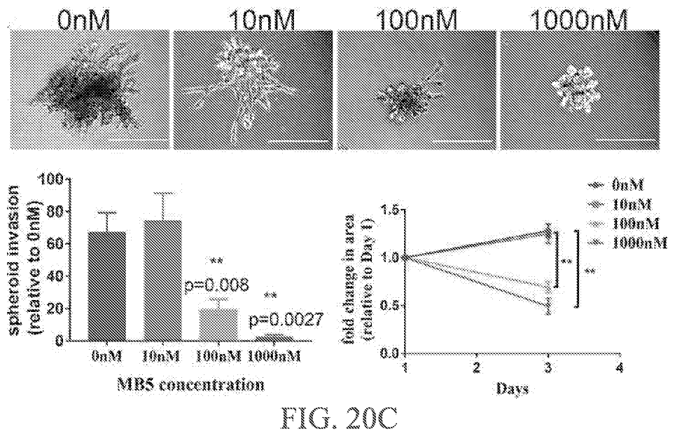

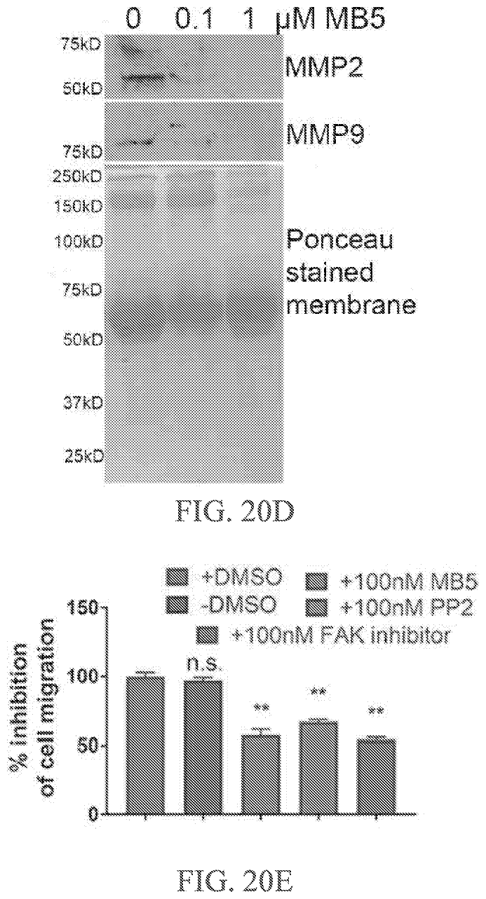

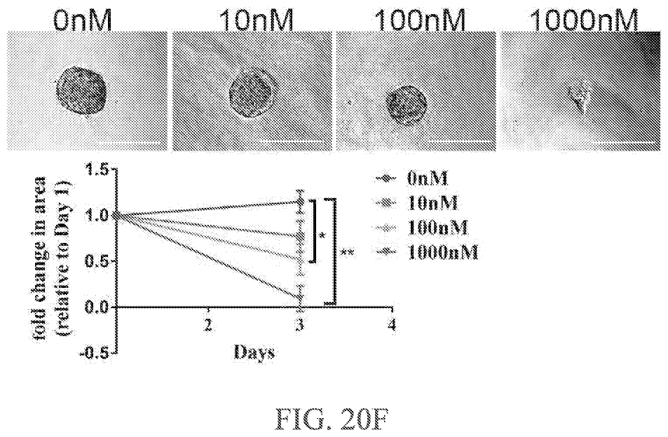

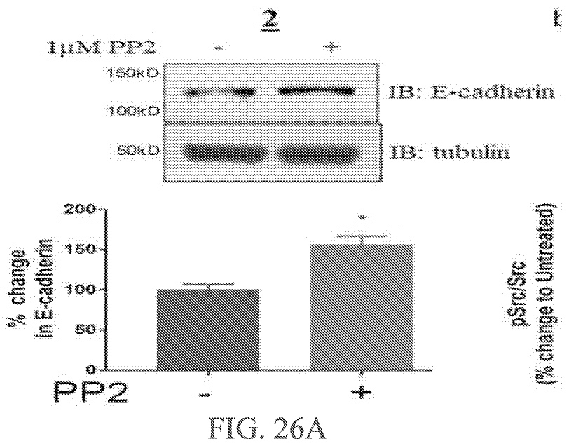

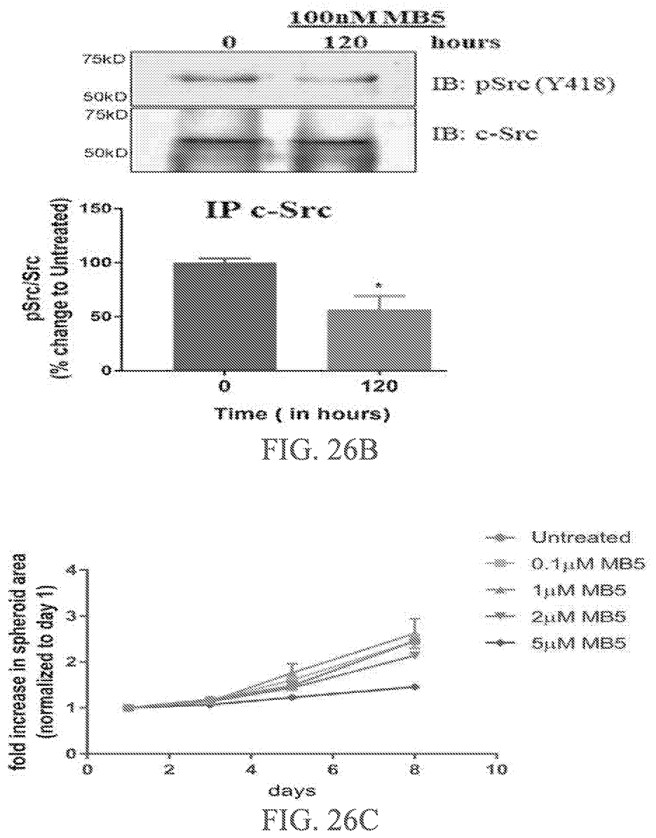

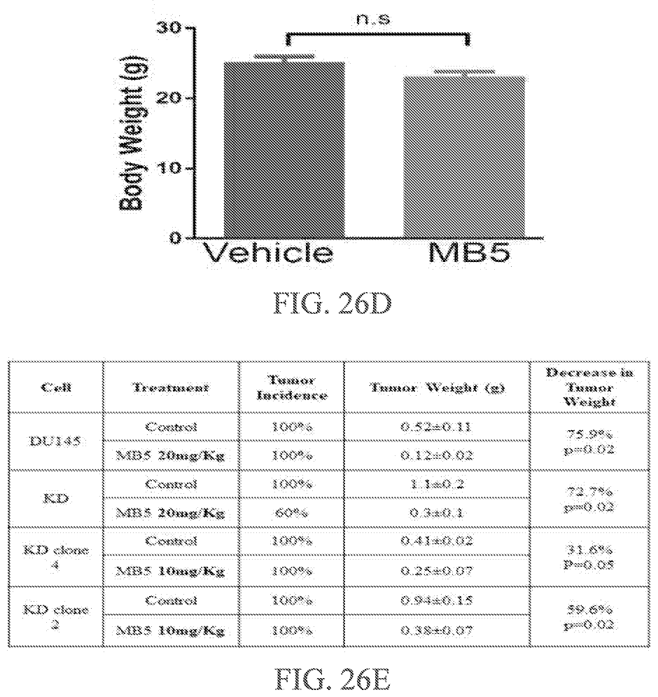

Next, it was tested whether MB5 as an inverse agonist of NKA receptor function could reverse EMT and stop invasion of PCa cells by rescuing α1 NKA expression in PCa. Subclone 5 and 2 cells were exposed to a long term treatment (0, 24, 48 and 72 hours) with 100 nM MB5. A significant time-dependent increase in α1 NKA and E-cadherin expression, along with a decreased expression of mesenchymal markers SNAIL and ZEB1 was observed, with maximal inhibition occurring at 72 hours (FIG. 20A). This was associated with a significant decrease in Src, FAK activation and Myc expression. Treatment of subclone 2 cells with the Src kinase inhibitor PP2 also resulted in increase in E-cadherin expression (FIG. 26A), suggesting that Src kinase activation might be partially responsible for E-cadherin loss through proteasomal cleavage in addition to transcriptional downregulation as observed in FIG. 16H. Moreover, expression of PCNA, a cell proliferation marker, was either abolished (in subclone 5) or significantly reduced (in subclone 2) by 72 hours, indicating the potential for MB5 to reduce cancer cell growth. Increase in E-cadherin and occludin expression by MB5 was further confirmed by immunostaining in subclone 2 cells treated with 1 μM MB5 for 24 hours (FIG. 20B).

Functionally, MB5 suppressed spheroid invasion by subclone 2 cells in a dose-dependent manner and significantly reduced spheroid size by 72 hours (FIG. 20C). This anti-invasive effect was further verified by Western blot analyses of media (FIG. 20D), which showed that MB5 treatment significantly reduced MMP2 and 9 secretions by subclone 2 spheroids. Finally, Boyden chamber assay (FIG. 20E) showed that pretreatment with 100 nM MB5 for 24 hours was sufficient to reduce cell migration by 50%, an effect that was comparable to Src or FAK inhibitor treatment. MB5 also significantly reduced the size of subclone 5 spheroids in 3D culture (FIG. 20F) in agreement with its inhibitory effect on PCNA. Taken together, the above studies indicate a molecular mechanism whereby increased surface expression of α1 NKA reigns in Src kinase activity in cancer cells, thus rescuing cell adhesion proteins like E-cadherin from proteasomal degradation and gradually suppressing the EMT phenotype.

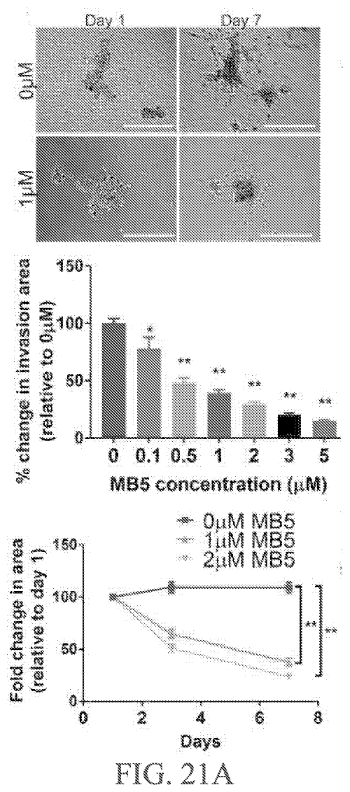

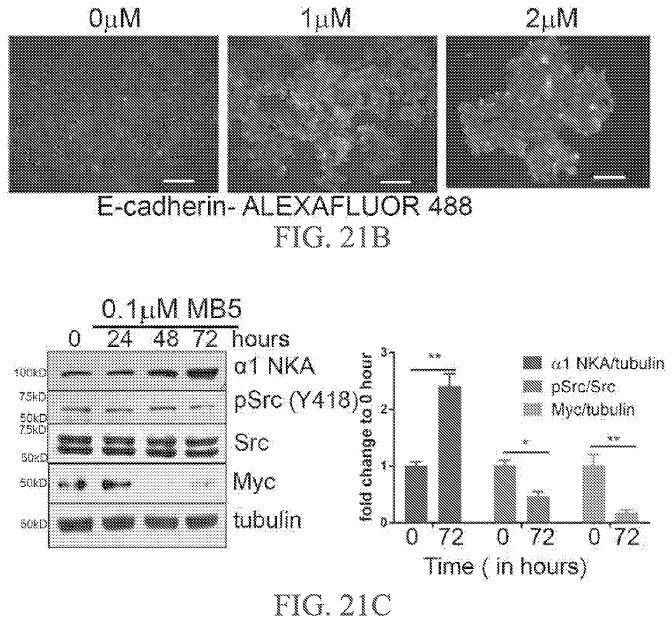

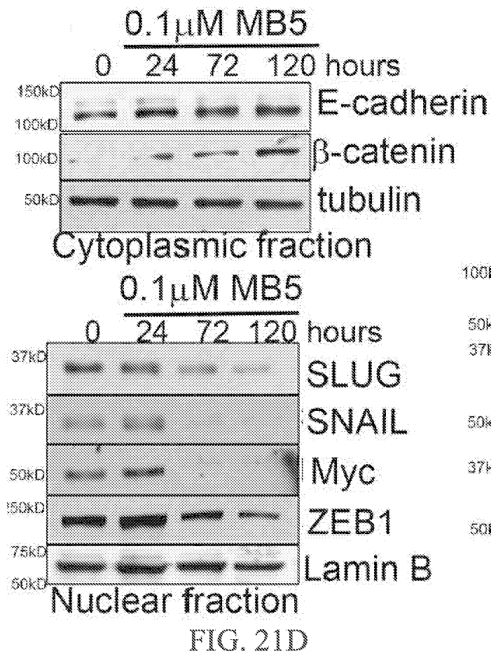

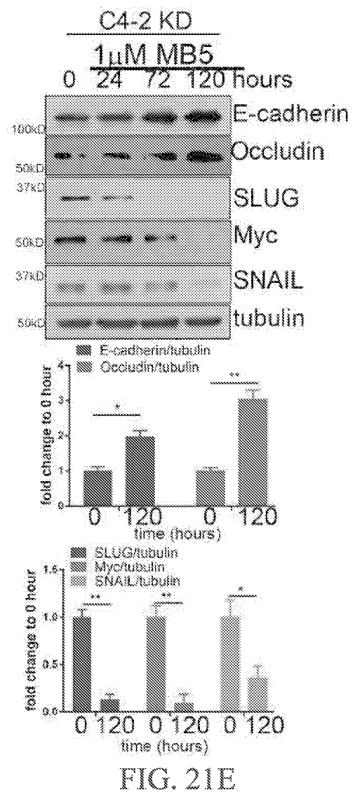

Because subclones 2 and 5 were selected and derived in the laboratory as models based on metastatic potentials and NKA expression levels, the universality of proposed MB5 mechanism and efficacy on NKA signaling, EMT, and invasiveness of PCa cells was further tested independently in PC3 and C4-2 cells. In 3D culture, vehicle-treated PC3 cells formed loose heterogeneous spheroids, which invaded into the matrix significantly by day 7. MB5 treatment suppressed spheroid invasion, and also reduced spheroid size in a concentration-dependent manner (about 25% inhibition at 0.1 μM and 50% at 1 μM; FIG. 21A). In ultralow attachment plates, control PC3 cells did not form cellular aggregates due to a null mutation of α-catenin gene, in accordance with previous reports. Nonetheless, MB5 treatment increased cell aggregation in a concentration-dependent manner and also significantly increased E-cadherin expression in those cells (FIG. 21B), confirming that MB5 works by tightening cell-cell attachment. Finally, Western blot and cell fractionation analyses (FIGS. 21C-21D) confirmed that 72-120 hours of MB5 treatment in PC3 cells significantly increased α1 NKA expression, decreased Src activation, and reversed the EMT phenotype. MB5 treatment was also able to upregulate occludin and E-cadherin expression in KD cells from C4-2, while inhibiting the expression of SNAIL, SLUG and Myc (FIG. 21E) along with Src activation (FIG. 26B). On the other hand, MB5 treatment at concentrations as high as 0.1-2 μM (FIG. 26C) did not significantly affect spheroid growth of the parental C4-2 cells, consistent with low α1 NKA endocytosis in these cells (FIG. 17B).

### Example 10—MB5 Reduces Tumor Growth in the Xenograft NOD/SCID Mouse Model

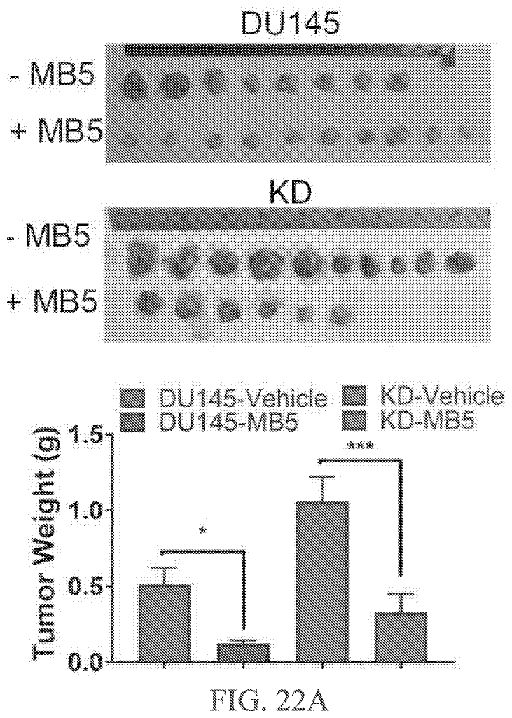

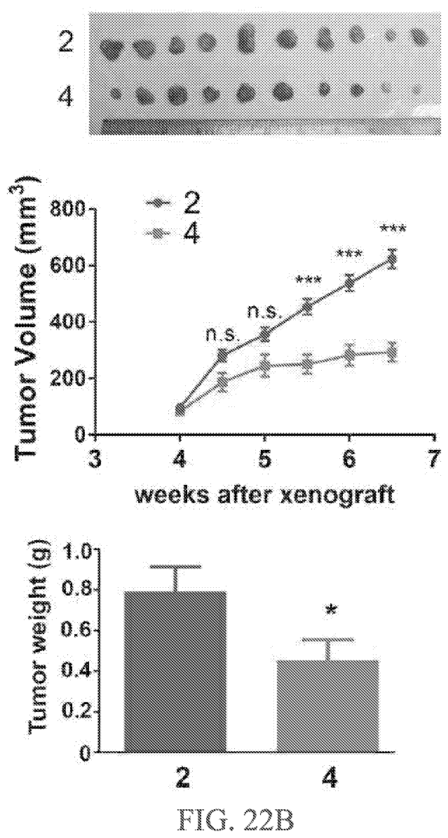

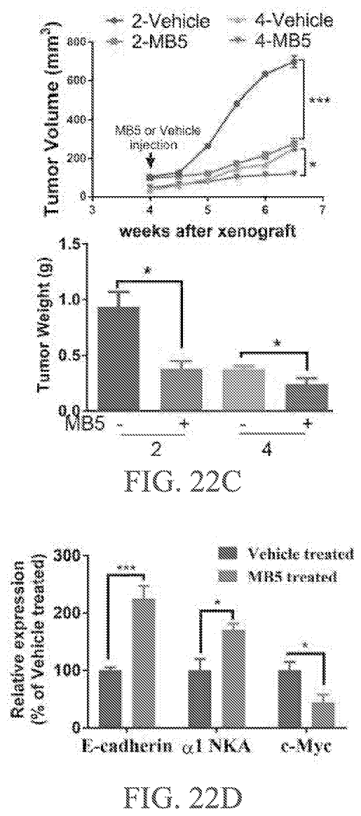

The therapeutic potential of MB5 in a tumor xenograft model was first tested by injecting DU145 or corresponding α1 knockdown cells (KD) into the right or left flank of 10-week-old NOD/SCID mice. The tumor growth was monitored twice weekly. Once the tumors reached a volume of approximately 100 mm³, the animals received daily injections of MB5 at 20 mg/kg intraperitoneally for 3.5 weeks. MB5 treatment significantly reduced (about 70%) tumor growth in both groups as shown in FIG. 22A, without significantly affecting bodyweight (FIG. 26D). Next, it was determined whether MB5 could decrease tumor growth of highly aggressive subclones derived from DU145. As shown in FIG. 22B, sub-clone 2 formed significantly larger tumors than sub-clone 4 when xenografted into NOD/SCID mice. MB5 treatment (10 mg/kg) significantly reduced tumor growth from both cell types (FIG. 22C). Furthermore, analyses of subclone 2 tumor lysates (FIG. 22D) confirmed that MB5 worked by increasing α1 NKA and E-cadherin expression. FIG. 26E summarizes the effect of MB5 treatment on all types of xenografted tumor growth from DU145 and derivative cell lines.

Discussion of Examples 5-10.

Molecular targets and therapeutics approaches focused on EMT have a high potential in the treatment of metastatic PCa. This study provides evidence that a reduction of al NKA polypeptide is sufficient to induce EMT, increase invasiveness and consequently aggressiveness of PCa. Upregulation of α1 NKA through gene-overexpression is sufficient to reduce tumor growth in the mouse NOD/SCID model, validating α1 NKA expression as a novel target in PCa. Mechanistically, increased endocytosis through activation of α1 NKA/Src signalosome complex is identified as the mechanism underlying the post translational downregulation of NKA in cancer cells. High throughput screening and pharmacological characterization identified the small molecule MB5 as a novel inverse agonist of α1 NKA/Src receptor complex, blocker of ouabain-induced signal transduction and α1 NKA endocytosis at far lower concentrations than those required to significantly inhibit NKA enzyme function (IC50 for Na/K-ATPase activity=10 μM). MB5 effectively reversed EMT and reduced metastatic potential of PCa cells in 3D culture and tumor growth in mouse tumor xenograft model.

Mechanistically, it was believed that tumor suppressor α1 NKA act as guardian of the upstream signaling pathways by regulating Src kinase, a protein that is required for receptor tyrosine kinase signaling. In several PCa models, this regulation is attenuated because of increased endocytosis of α1 NKA It was shown here that the progressive loss of α1 NKA further aggravates PCa phenotype by promoting EMT through direct inhibition of E-cadherin and occludin expression and dissolution of cell-cell junction. Evidence from both cell and animal models indicate that the loss of E-cadherin promotes tumor progression, invasion and metastasis. It was found that α1 NKA is a critical regulator of E-cadherin expression in PCa. This regulation most likely occurred at both transcriptional and post translational level. First, α1 NKA downregulation resulted in an increase in Src activity, which could enhance the endocytosis and degradation of E-cadherin. This is also consistent with the data presented in FIG. 26A. A second level of regulation may come through transcriptional regulators such as ZEB1 and SNAIL that are known repressors of E-cadherin transcription. Although the exact mechanism is cell-specific, there was a generalized upregulation of mesenchymal markers combined with decrease in adherens junction proteins in all PCa cell lines studied.

Several studies have indicated that NKA can itself function as a cell-cell attachment molecule through NKA R subunit/P subunit interaction between adjacent cells. Specifically, treatment of LLC-PK1 cells with TGFβ was previously shown to induce an EMT phenotype by downregulating p subunit expression through a post translational mechanism. However, the R subunit itself does not have any known catalytic or signaling function. It was believed that activation of α1 NKA/Src signaling complex in cancer cells contributes to its decreased expression at the plasma membrane. Factors that are common in the tumor microenvironment, such as hypoxia/oxidative stress, can induce endocytosis of the NKA through a NKA/Src-dependent feedforward mechanism known as the NKA amplification loop. Furthermore, increased extracellular potassium released from apoptotic and necrotic cancer cells can also stabilize the α1 NKA/Src signaling complex in an active state similar to the one stabilized by ouabain, and thereby promote endocytosis. It was therefore proposed that this can activate multiple oncogenic signaling pathways and also lead to weakened cell-cell attachment by downregulating β-β interaction (FIGS. 23A-23B).

In this respect, MB5 as an inverse agonist of the receptor α1 NKA/Src, potently blocked the endocytosis and increased the surface expression of α1 NKA in PCa cells. MB5 was also effective in reversing EMT phenotype by upregulation of E-cadherin and down-regulation of mesenchymal markers SNAIL, SLUG and ZEB1. Consequently, it inhibited the growth and invasiveness of PCa spheroids. Finally, xenograft studies confirmed that MB5 effectively reduced tumor growth of PCa. Two aspects of this new discovery are noted. First, MB5 represents the first class of inverse agonists of receptor NKA/Src complex. The findings demonstrate the need and feasibility for developing other potent, effective and structurally diverse classes of inverse agonists targeting the α1 NKA/Src signaling complex. Moreover, MB5 could serve as a prototype to generate potential anti-cancer drug candidates. Second, in addition to PCa, the loss of α1 NKA occurs in several other types of epithelia-derived tumors.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Brawley, O. W. Trends in prostate cancer in the United States. J Natl Cancer Inst Monogr 2012, 2012, 152-156, doi:10.1093/jncimonographs/lgs035.
2. Bashir, M. N. Epidemiology of Prostate Cancer. Asian Pac J Cancer Prev 2015, 16, 5137-5141, doi:10.7314/apjcp.2015.16.13.5137.
3. Kalluri, R.; Weinberg, R. A. The basics of epithelial-mesenchymal transition. J Clin Invest 2009, 119, 1420-1428, doi:10.1172/JC139104.
4. Lambert, A. W.; Pattabiraman, D. R.; Weinberg, R. A. Emerging Biological Principles of Metastasis. Cell 2017, 168, 670-691, doi:10.1016/j.cell.2016.11.037.
5. Skou, J. C. The (Na++K+)-ATPase: coupling of the reaction with ATP to the reaction with Na+ and K+. Ann N Y Acad Sci 1982, 402, 169-184.
6. Xie, J.; Ye, Q.; Cui, X.; Madan, N.; Yi, Q.; Pierre, S. V.; Xie, Z. Expression of rat Na-K-ATPase alpha2 enables ion pumping but not ouabain-induced signaling in alpha1-deficient porcine renal epithelial cells. Am J Physiol Cell Physiol 2015, 309, C373-382, doi:10.1152/ajpcell.00103.2015.
7. Madan, N.; Xu, Y.; Duan, Q.; Banerjee, M.; Larre, I.; Pierre, S. V.; Xie, Z. Src-independent ERK signaling through the rat alpha3 isoform of Na/K-ATPase. Am J Physiol Cell Physiol 2017, 312, C222-C232, doi:10.1152/ajpcell.00199.2016.
8. Wang X, C. L., Xie J X, Cui X, Zhang J et al. A Caveolin Binding Motif in Na/K-ATPase is Required for Stem Cell Differentiation and Organogenesis in Mammals and C. elegans Sci Adv 2020, in press.
9. Cui, X.; Xie, Z. Protein Interaction and Na/K-ATPase-Mediated Signal Transduction. Molecules 2017, 22, doi:10.3390/molecules22060990.
10. Rajasekaran, S. A.; Palmer, L. G.; Quan, K.; Harper, J. F.; Ball, W. J., Jr.; Bander, N. H.; Peralta Soler, A.; Rajasekaran, A. K. Na,K-ATPase beta-subunit is required for epithelial polarization, suppression of invasion, and cell motility. Mol Biol Cell 2001, 12, 279-295, doi:10.1091/mbc.12.2.279.
11. Shoshani, L.; Contreras, R. G.; Roldan, M. L.; Moreno, J.; Lazaro, A.; Balda, M. S.; Matter, K.; Cereijido, M. The polarized expression of Na+,K+-ATPase in epithelia depends on the association between beta-subunits located in neighboring cells. Mol Biol Cell 2005, 16, 1071-1081, doi:10.1091/mbc.e04-03-0267.
12. Rajasekaran, S. A.; Huynh, T. P.; Wolle, D. G.; Espineda, C. E.; Inge, L. J.; Skay, A.; Lassman, C.; Nicholas, S. B.; Harper, J. F.; Reeves, A. E., et al. Na,K-ATPase subunits as markers for epithelial-mesenchymal transition in cancer and fibrosis. Mol Cancer Ther 2010, 9, 1515-1524, doi:10.1158/1535-7163.MCT-09-0832.
13. Lovisa, S.; LeBleu, V. S.; Tampe, B.; Sugimoto, H.; Vadnagara, K.; Carstens, J. L.; Wu, C. C.; Hagos, Y.; Burckhardt, B. C.; Pentcheva-Hoang, T., et al. Epithelial-to-mesenchymal transition induces cell cycle arrest and parenchymal damage in renal fibrosis. Nat Med 2015, 21, 998-1009, doi:10.1038/nm.3902.
14. Baker Bechmann, M.; Rotoli, D.; Morales, M.; Maeso Mdel, C.; Garcia Mdel, P.; Avila, J.; Mobasheri, A.; Martin-Vasallo, P. Na,K-ATPase Isozymes in Colorectal Cancer and Liver Metastases. Front Physiol 2016, 7, 9, doi:10.3389/fphys.2016.00009.
15. Banerjee, M.; Cui, X.; Li, Z.; Yu, H.; Cai, L.; Jia, X.; He, D.; Wang, C.; Gao, T.; Xie, Z. Na/K-ATPase Y260 Phosphorylation-mediated Src Regulation in Control of Aerobic Glycolysis and Tumor Growth. Sci Rep 2018, 8, 12322, doi:10.1038/s41598-018-29995-2.
16. Li, Z.; Zhang, Z.; Xie, J. X.; Li, X.; Tian, J.; Cai, T.; Cui, H.; Ding, H.; Shapiro, J. I.; Xie, Z. Na/K-ATPase mimetic pNaKtide peptide inhibits the growth of human cancer cells. J Biol Chem 2011, 286, 32394-32403, doi:10.1074/jbc.M110.207597.
17. Laursen, M.; Gregersen, J. L.; Yatime, L.; Nissen, P.; Fedosova, N. U. Structures and characterization of digoxin- and bufalin-bound Na+,K+-ATPase compared with the ouabain-bound complex. Proc Natl Acad Sci USA 2015, 112, 1755-1760, doi:10.1073/pnas.1422997112.
18. Ye, Q.; Li, Z.; Tian, J.; Xie, J. X.; Liu, L.; Xie, Z. Identification of a potential receptor that couples ion transport to protein kinase activity. J Biol Chem 2011, 286, 6225-6232, doi:10.1074/jbc.M110.202051.
19. Ye, Q.; Lai, F.; Banerjee, M.; Duan, Q.; Li, Z.; Si, S.; Xie, Z. Expression of mutant alpha1 Na/K-ATPase defective in conformational transition attenuates Src-mediated signal transduction. J Biol Chem 2013, 288, 5803-5814, doi:10.1074/jbc.M112.442608.
20. Lai, F.; Madan, N.; Ye, Q.; Duan, Q.; Li, Z.; Wang, S.; Si, S.; Xie, Z. Identification of a mutant alpha1 Na/K-ATPase that pumps but is defective in signal transduction. J Biol Chem 2013, 288, 13295-13304, doi:10.1074/jbc.M113.467381.
21. Banerjee, M.; Duan, Q.; Xie, Z. SH2 Ligand-Like Effects of Second Cytosolic Domain of Na/K-ATPase alpha1 Subunit on Src Kinase. PLoS One 2015, 10, e0142119, doi:10.1371/journal.pone.0142119.
22. Yan, Y.; Shapiro, A. P.; Haller, S.; Katragadda, V.; Liu, L.; Tian, J.; Basrur, V.; Malhotra, D.; Xie, Z. J.; Abraham, N. G., et al. Involvement of reactive oxygen species in a feed-forward mechanism of Na/K-ATPase-mediated signaling transduction. J Biol Chem 2013, 288, 34249-34258, doi:10.1074/jbc.M113.461020.
23. Liu, J.; Kesiry, R.; Periyasamy, S. M.; Malhotra, D.; Xie, Z.; Shapiro, J. I. Ouabain induces endocytosis of plasmalemmal Na/K-ATPase in LLC-PK1 cells by a clathrin-dependent mechanism. Kidney Int 2004, 66, 227-241, doi:10.1111/j.1523-1755.2004.00723.x.
24. Liu, L.; Abramowitz, J.; Askari, A.; Allen, J. C. Role of caveolae in ouabain-induced proliferation of cultured vascular smooth muscle cells of the synthetic phenotype. Am J Physiol Heart Circ Physiol 2004, 287, H2173-2182, doi:10.1152/ajpheart.00352.2004.
25. Liu, L.; Li, J.; Liu, J.; Yuan, Z.; Pierre, S. V.; Qu, W.; Zhao, X.; Xie, Z. Involvement of Na+/K+-ATPase in hydrogen peroxide-induced hypertrophy in cardiac myocytes. Free Radic Biol Med 2006, 41, 1548-1556, doi:10.1016/j.freeradbiomed.2006.08.018.
26. Zhang, Z.; Li, Z.; Tian, J.; Jiang, W.; Wang, Y.; Zhang, X.; Li, Z.; You, Q.; Shapiro, J. I.; Si, S., et al. Identification of hydroxyxanthones as Na/K-ATPase ligands. Mol Pharmacol 2010, 77, 961-967, doi:10.1124/mol.110.063974.
27. Liang, M.; Tian, J.; Liu, L.; Pierre, S.; Liu, J.; Shapiro, J.; Xie, Z. J. Identification of a pool of non-pumping Na/K-ATPase. J Biol Chem 2007, 282, 10585-10593, doi:10.1074/jbc.M609181200.
28. Forte, E.; Chimenti, I.; Rosa, P.; Angelini, F.; Pagano, F.; Calogero, A.; Giacomello, A.; Messina, E. EMT/MET at the Crossroad of Stemness, Regeneration and Oncogenesis: The Ying-Yang Equilibrium Recapitulated in Cell Spheroids. Cancers (Basel) 2017, 9, doi:10.3390/cancers9080098.
29. Gialeli, C.; Theocharis, A. D.; Karamanos, N. K. Roles of matrix metalloproteinases in cancer progression and their pharmacological targeting. FEBS J 2011, 278, 16-27, doi:10.1111/j.1742-4658.2010.07919.x.
30. Tian, J.; Cai, T.; Yuan, Z.; Wang, H.; Liu, L.; Haas, M.; Maksimova, E.; Huang, X. Y.; Xie, Z. J. Binding of Src to Na+/K+-ATPase forms a functional signaling complex. Mol Biol Cell 2006, 17, 317-326, doi:10.1091/mbc.e05-08-0735.
31. Chibalin, A. V.; Katz, A. I.; Berggren, P. O.; Bertorello, A. M. Receptor-mediated inhibition of renal Na(+)-K(+)-ATPase is associated with endocytosis of its alpha- and beta-subunits. Am J Physiol 1997, 273, C1458-1465, doi:10.1152/ajpcell.1997.273.5.C1458.
32. Done, S. C.; Leibiger, I. B.; Efendiev, R.; Katz, A. I.; Leibiger, B.; Berggren, P. O.; Pedemonte, C. H.; Bertorello, A. M. Tyrosine 537 within the Na+,K+-ATPase alpha-subunit is essential for AP-2 binding and clathrin-dependent endocytosis. J Biol Chem 2002, 277, 17108-17111, doi:10.1074/jbc.M201326200.
33. Palacios, F.; Tushir, J. S.; Fujita, Y.; D'Souza-Schorey, C. Lysosomal targeting of E-cadherin: a unique mechanism for the down-regulation of cell-cell adhesion during epithelial to mesenchymal transitions. Mol Cell Biol 2005, 25, 389-402, doi:10.1128/MCB.25.1.389-402.2005.
34. Inge, L. J.; Rajasekaran, S. A.; Wolle, D.; Barwe, S. P.; Ryazantsev, S.; Ewing, C. M.; Isaacs, W. B.; Rajasekaran, A. K. alpha-Catenin overrides Src-dependent activation of beta-catenin oncogenic signaling. Mol Cancer Ther 2008, 7, 1386-1397, doi:10.1158/1535-7163.MCT-07-2029.
35. Onder, T. T.; Gupta, P. B.; Mani, S. A.; Yang, J.; Lander, E. S.; Weinberg, R. A. Loss of E-cadherin promotes metastasis via multiple downstream transcriptional pathways. Cancer Res 2008, 68, 3645-3654, doi:10.1158/0008-5472.CAN-07-2938.
36. Petrova, Y. I.; Schecterson, L.; Gumbiner, B. M. Roles for E-cadherin cell surface regulation in cancer. Mol Biol Cell 2016, 27, 3233-3244, doi:10.1091/mbc.E16-01-0058.
37. Sinkevicius, K. W.; Bellaria, K. J.; Barrios, J.; Pessina, P.; Gupta, M.; Brainson, C. F.; Bronson, R. T.; Kim, C. F. E-Cadherin Loss Accelerates Tumor Progression and Metastasis in a Mouse Model of Lung Adenocarcinoma. Am J Respir Cell Mol Biol 2018, 59, 237-245, doi:10.1165/rcmb.2017-02100C.
38. Behrens, J.; Vakaet, L.; Friis, R.; Winterhager, E.; Van Roy, F.; Mareel, M. M.; Birchmeier, W. Loss of epithelial differentiation and gain of invasiveness correlates with tyrosine phosphorylation of the E-cadherin/beta-catenin complex in cells transformed with a temperature-sensitive v-SRC gene. J Cell Biol 1993, 120, 757-766.
39. Sanchez-Tillo, E.; Lazaro, A.; Torrent, R.; Cuatrecasas, M.; Vaquero, E. C.; Castells, A.; Engel, P.; Postigo, A. ZEB1 represses E-cadherin and induces an EMT by recruiting the SWI/SNF chromatin-remodeling protein BRG1. Oncogene 2010, 29, 3490-3500, doi:10.1038/onc.2010.102.
40. Vagin, O.; Dada, L. A.; Tokhtaeva, E.; Sachs, G. The Na-K-ATPase alpha(1)beta(1) heterodimer as a cell adhesion molecule in epithelia. Am J Physiol Cell Physiol 2012, 302, C1271-1281, doi:10.1152/ajpcell.00456.2011.
41. Liu, J.; Shapiro, J. I. Regulation of sodium pump endocytosis by cardiotonic steroids: Molecular mechanisms and physiological implications. Pathophysiology 2007, 14, 171-181, doi:10.1016/j.pathophys.2007.09.008.
42. Dada, L. A.; Chandel, N. S.; Ridge, K. M.; Pedemonte, C.; Bertorello, A. M.; Sznajder, J. I. Hypoxia-induced endocytosis of Na,K-ATPase in alveolar epithelial cells is mediated by mitochondrial reactive oxygen species and PKC-zeta. J Clin Invest 2003, 111, 1057-1064, doi:10.1172/JCI16826.
43. Bogdanova, A.; Petrushanko, I. Y.; Hernansanz-Agustin, P.; Martinez-Ruiz, A. “Oxygen Sensing” by Na,K-ATPase: These Miraculous Thiols. Front Physiol 2016, 7, 314, doi:10.3389/fphys.2016.00314.
44. Comellas, A. P.; Dada, L. A.; Lecuona, E.; Pesce, L. M.; Chandel, N. S.; Quesada, N.; Budinger, G. R.; Strous, G. J.; Ciechanover, A.; Sznajder, J. I. Hypoxia-mediated degradation of Na,K-ATPase via mitochondrial reactive oxygen species and the ubiquitin-conjugating system. Circ Res 2006, 98, 1314-1322, doi:10.1161/01.RES.0000222418.99976.1d.
45. Eil, R.; Vodnala, S. K.; Clever, D.; Klebanoff, C. A.; Sukumar, M.; Pan, J. H.; Palmer, D. C.; Gros, A.; Yamamoto, T. N.; Patel, S. J., et al. Ionic immune suppression within the tumour microenvironment limits T cell effector function. Nature 2016, 537, 539-543, doi:10.1038/nature19364.
46. Espineda, C.; Seligson, D. B.; James Ball, W., Jr.; Rao, J.; Palotie, A.; Horvath, S.; Huang, Y.; Shi, T.; Rajasekaran, A. K. Analysis of the Na,K-ATPase alpha- and beta-subunit expression profiles of bladder cancer using tissue microarrays. Cancer 2003, 97, 1859-1868, doi:10.1002/cncr.11267.
47. Liang, M.; Cai, T.; Tian, J.; Qu, W.; Xie, Z. J. Functional characterization of Src-interacting Na/K-ATPase using RNA interference assay. J Biol Chem 2006, 281, 19709-19719, doi:10.1074/jbc.M512240200.
48. Golubovskaya, V. M.; Nyberg, C.; Zheng, M.; Kweh, F.; Magis, A.; Ostrov, D.; Cance, W. G. A small molecule inhibitor, 1,2,4,5-benzenetetraamine tetrahydrochloride, targeting the y397 site of focal adhesion kinase decreases tumor growth. J Med Chem 2008, 51, 7405-7416, doi:10.1021/jm800483v.
49. Berens, E. B.; Holy, J. M.; Riegel, A. T.; Wellstein, A. A Cancer Cell Spheroid Assay to Assess Invasion in a 3D Setting. J Vis Exp 2015, 10.3791/53409, doi:10.3791/53409.

It will be understood that various details of the presently disclosed subject matter can be changed without departing from the scope of the subject matter disclosed herein. Furthermore, the foregoing description is for the purpose of illustration only, and not for the purpose of limitation.

1. A compound, comprising the formula (I): 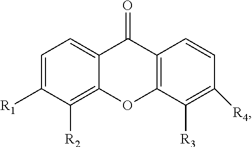
wherein R₁, R₂, R₃, and R₄ are independently selected from a hydrogen atom, a hydroxyl, an amine, a sulfonic acid, a thiol, a fluorine atom, or a phosphate group,
or wherein R₁ and R₂ are a hydrogen and R₃ and R₄ are combined to form a heterocyclic group including one or more nitrogen atoms.

2. The compound of claim 1, wherein the compound has the following formula (II):

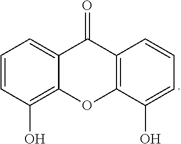 [SMILES: O=c1c2cccc(O)c2oc2c(O)cccc12]

3. The compound of claim 1, wherein the compound has the following formula (III):

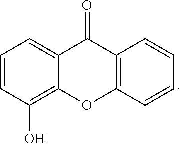 [SMILES: O=c1c2ccccc2oc2c(O)cccc12]

4. The compound of claim 1, wherein the compound has the following formula (IV):

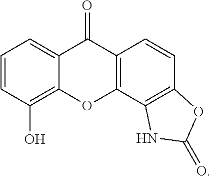 [SMILES: O=c1[nH]c2c(ccc3c(=O)c4cccc(O)c4oc32)o1]

5. The compound of claim 1, wherein the compound has the following formula (V):

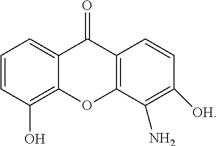 [SMILES: Nc1c(O)ccc2c(=O)c3cccc(O)c3oc12]

6. The compound of claim 1, wherein the compound has the following formula (VI):

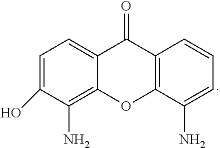 [SMILES: Nc1cccc2c(=O)c3ccc(O)c(N)c3oc12]

7. The compound of claim 1, wherein the compound has the following formula (VII):

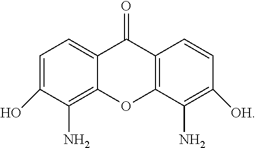 [SMILES: Nc1c(O)ccc2c(=O)c3ccc(O)c(N)c3oc12]

8. The compound of claim 1, wherein the compound has the following formula (VIII):

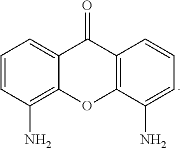 [SMILES: Nc1cccc2c(=O)c3cccc(N)c3oc12]

9. The compound of claim 1, wherein the compound has the following formula (IX):

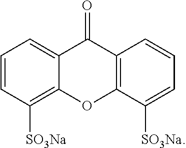

10. The compound of claim 1, wherein the compound has the following formula (X):

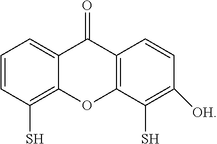 [SMILES: O=c1c2cccc(S)c2oc2c(S)c(O)ccc12]

11. The compound of claim 1, wherein the compound has the following formula (XI):

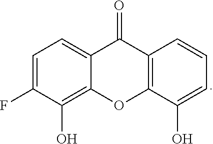 [SMILES: O=c1c2cccc(O)c2oc2c(O)c(F)ccc12]

12. The compound of claim 1, wherein the compound has the following formula (XII):

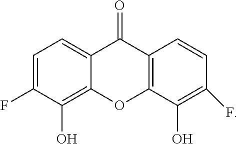 [SMILES: O=c1c2ccc(F)c(O)c2oc2c(O)c(F)ccc12]

13. The compound of claim 1, wherein the compound has the following formula (XIII):

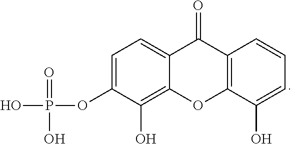 [SMILES: O=c1c2cccc(O)c2oc2c(O)c(OP(=O)(O)O)ccc12]

14. A compound having the formula selected from the group consisting of:

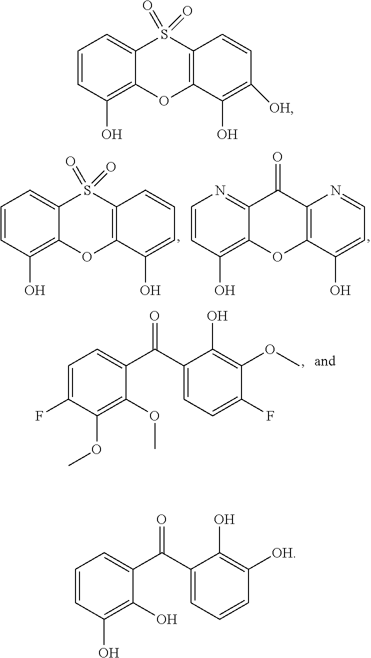

15. A pharmaceutical composition, comprising a compound of claim 1, and a pharmaceutically-acceptable vehicle, carrier, or excipient.

16. A method of treating cancer, comprising administering to a subject an effective amount of a compound according to claim 1.

17. The method of claim 16, wherein the cancer is a primary cancer.

18. The method of claim 16, wherein the cancer is a secondary cancer.

19. The method of claim 16, wherein the subject has cancer.

20. The method of claim 16, wherein the compound is administered in an amount sufficient to reduce an endocytosis of an α1 Na/K-ATPase in a cancer cell.

21. The method of claim 16, wherein the compound has the following formula (XIV):

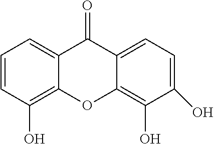 [SMILES: O=c1c2cccc(O)c2oc2c(O)c(O)ccc12]

22. The method of claim 16, wherein the compound has the following formula (XV): 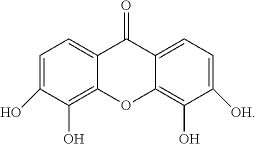 [SMILES: O=c1c2ccc(O)c(O)c2oc2c(O)c(O)ccc12]
